(19) United States
(12) Patent Application Publication
(10) Pub. No.: US 2017/0281612 A1
(43) Pub. Date: Oct. 5, 2017

(54) Methods for the Treatment of Cystic Fibrosis

(71) Applicant: Nivalis Therapeutics, Inc., Boulder, CO (US)

(72) Inventors: Sherif Gabriel, Chapel Hill, NC (US); Peter Bove, Longmont, CO (US)

(21) Appl. No.: 15/510,482

(22) PCT Filed: Oct. 8, 2015
(86) PCT No.: PCT/US15/54728

§ 371 Date: Mar. 10, 2017

Related U.S. Application Data
(60) Provisional application No. 62/061,557, filed on Oct. 8, 2014.
(60) Provisional application No. 62/093,712, filed on Dec. 18, 2014.
(60) Provisional application No. 62/138,792, filed on Mar. 26, 2015.
(60) Provisional application No. 62/209,724, filed on Aug. 25, 2015.

(51) Int. Cl. A61K 31/47; A61K 31/443

(52) U.S. Cl. CPC A61K 31/47; A61K 31/443

(57) ABSTRACT

The present invention is directed to a method of treating or lessening the severity of cystic fibrosis in a patient, comprising the step of administering to said patient an effective amount of an inhibitor of S-nitrosoglutathione reductase (GSNOR) in combination with one or more secondary active agents.

### FIELD OF THE INVENTION

The present invention is directed to a method of treating or lessening the severity of cystic fibrosis in a patient, comprising the step of administering to said patient an effective amount of an inhibitor of S-nitrosoglutathione reductase (GSNOR) in combination with one or more secondary active agents.

### BACKGROUND

Cystic fibrosis (CF) is one of the most common lethal genetic diseases in Caucasians. Approximately one in 3,500 children in the US is born with CF each year. It is a disease that affects all racial and ethnic groups, but is more common among Caucasians. An estimated 30,000 American adults and children have CF, and the median predicted age of survival is 37.4 years (CFF Registry Report 2007, Cystic Fibrosis Foundation, Bethesda, Md.). CF is an autosomal recessive hereditary disease caused by a mutation in the gene for the cystic fibrosis transmembrane regulator (CFTR) protein. CF is diagnosed by the level of chloride in sweat because patients with CF have elevated sweat chloride due to the primary defect in CFTR. More than 1,000 disease-associated mutations have been discovered in the CFTR gene with the most common mutation being a deletion of the amino acid phenylalanine at position 508 (F508del). This defect is present in 70% of CF patients. The CFTR protein is located on the apical membrane and is responsible for chloride transport across epithelial cells on mucosal surfaces. S-nitrosoglutathione (GSNO) has been identified as a positive modulator of CFTR. As S-nitrosoglutathione reductase (GSNOR) is the primary catabolizing enzyme of GSNO, it is hypothesized that inhibition of GSNOR may improve F508del-CFTR function via nitrosation of chaperone proteins, prevention of CFTR proteosomal degradation, promotion of CFTR maturation, and maintainence of epithelial tight junctions. Currently there is no curative treatment for CF; therefore, new therapies are needed for the disease.

GSNO is a key regulator of nitric oxide (NO) homeostasis and cellular S-nitrosothiol (SNO) levels, and studies have focused on examining endogenous production of GSNO and SNO proteins, which occurs downstream from the production of the NO radical by the nitric oxide synthetase (NOS) enzymes. More recently there has been an increasing understanding of enzymatic catabolism of GSNO which has an important role in governing available concentrations of GSNO and consequently available NO and SNO's.

NO is one of the few gaseous signaling molecules known in biological systems, and plays an important role in controlling various biological events. For example, the endothelium uses NO to signal surrounding smooth muscle in the walls of arterioles to relax, resulting in vasodilation and increased blood flow to hypoxic tissues. NO is also involved in regulating smooth muscle proliferation, platelet function, and neurotransmission, and plays a role in host defense. Although NO is highly reactive and has a lifetime of a few seconds, it can both diffuse freely across membranes and bind to many molecular targets. These attributes make NO an ideal signaling molecule capable of controlling biological events between adjacent cells and within cells.

NO is a free radical gas, which makes it reactive and unstable, thus NO is short lived in vivo, having a half life of 3-5 seconds under physiologic conditions. In the presence of oxygen, NO can combine with thiols to generate a biologically important class of stable NO adducts called S-nitrosothiols (SNO's). This stable pool of NO has been postulated to act as a source of bioactive NO and as such appears to be critically important in health and disease, given the centrality of NO in cellular homeostasis (Stamler et al., Proc. Natl. Acad. Sci. USA, 89:7674-7677 (1992)). Protein SNO's play broad roles in the function of cardiovascular, respiratory, metabolic, gastrointestinal, immune, and central nervous system (Foster et al., Trends in Molecular Medicine, 9 (4):160-168, (2003)). One of the most studied SNO's in biological systems is S-nitrosoglutathione (GSNO) (Gaston et al., Proc. Natl. Acad. Sci. USA 90:10957-10961 (1993)), an emerging key regulator in NO signaling since it is an efficient trans-nitrosating agent and appears to maintain an equilibrium with other S-nitrosated proteins (Liu et al., Nature, 410:490-494 (2001)) within cells. Given this pivotal position in the NO-SNO continuum, GSNO provides a therapeutically promising target to consider when NO modulation is pharmacologically warranted.

Central to this understanding of GSNO catabolism, researchers have recently identified a highly conserved S-nitrosoglutathione reductase (GSNOR) (Jensen et al., Biochem J., 331:659-668 (1998); Liu et al., (2001)). GSNOR is also known as glutathione-dependent formaldehyde dehydrogenase (GSH-FDH), alcohol dehydrogenase 3 (ADH-3) (Uotila and Koivusalo, Coenzymes and Cofactors., D. Dolphin, ed. pp. 517-551 (New York, John Wiley & Sons, (1989)), and alcohol dehydrogenase 5 (ADH-5). Importantly GSNOR shows greater activity toward GSNO than other substrates (Jensen et al., (1998); Liu et al., (2001)) and appears to mediate important protein and peptide denitrosating activity in bacteria, plants, and animals. GSNOR appears to be the major GSNO-metabolizing enzyme in eukaryotes (Liu et al., (2001)). Thus, GSNO can accumulate in biological compartments where GSNOR activity is low or absent (e.g., airway lining fluid) (Gaston et al., (1993)).

GSNO specifically has been implicated in physiologic processes ranging from the drive to breathe (Lipton et al., Nature, 413:171-174 (2001)), to regulation of the cystic fibrosis transmembrane regulator (Zaman et al., Biochem Biophys Res Commun, 284:65-70 (2001)), to regulation of vascular tone, thrombosis, and platelet function (de Belder et al., Cardiovasc Res.; 28(5):691-4 (1994)), Z. Kaposzta, et al., Circulation; 106(24): 3057-3062, (2002)) as well as host defense (de Jesus-Berrios et al., Curr. Biol., 13:1963-1968 (2003)). Other studies have found that GSNOR protects yeast cells against nitrosative stress both in vitro (Liu et al., (2001)) and in vivo (de Jesus-Berrios et al., (2003)).

Collectively, data suggest GSNO as a primary physiological ligand for the enzyme S-nitrosoglutathione reductase (GSNOR), which catabolizes GSNO and consequently reduces available SNO's and NO in biological systems (Liu et al., (2001)), (Liu et al., Cell, 116(4), 617-628 (2004)), and (Que et al., Science, 308, (5728):1618-1621 (2005)). As such, this enzyme plays a central role in regulating local and systemic bioactive NO. Since perturbations in NO bioavailability has been linked to the pathogenesis of numerous disease states, including hypertension, atherosclerosis, thrombosis, pulmonary disorders, gastrointestinal disorders, inflammation, and cancer, agents that regulate GSNOR activity are candidate therapeutic agents for treating diseases associated with NO imbalance.

Nitric oxide (NO), S-nitrosoglutathione (GSNO), and S-nitrosoglutathione reductase (GSNOR) regulate normal lung physiology and contribute to lung pathophysiology. Under normal conditions, NO and GSNO maintain normal lung physiology and function via their anti-inflammatory and bronchodilatory actions. Lowered levels of these mediators in pulmonary diseases such as asthma, chronic obstructive pulmonary disease (COPD), and cystic fibrosis may occur via up-regulation of GSNOR enzyme activity. These lowered levels of NO and GSNO, and thus lowered anti-inflammatory capabilities, are key events that contribute to pulmonary diseases and which can potentially be reversed via GSNOR inhibition.

S-nitrosoglutathione (GSNO) has been shown to promote repair and/or regeneration of mammalian organs, such as the heart (Lima et al., 2010), blood vessels (Lima et al., 2010) skin (Georgii et al., 2010), eye or ocular structures (Haq et al., 2007) and liver (Prince et al., 2010). S-nitrosoglutathione reductase (GSNOR) is the major catabolic enzyme of GSNO. Inhibition of GSNOR is thought to increase endogenous GSNO, thus promoting organ repair and regeneration.

NO, GSNO, and GSNOR can also exert influences on disorders of the gastrointestinal (GI) tract, such as inflammatory bowel disease (IBD) (including Crohn's and ulcerative colitis) and possibly cystic fibrosis gastrointestinal disease. Under normal conditions, NO and GSNO function to maintain normal intestinal physiology via anti-inflammatory actions and maintenance of the intestinal epithelial cell barrier.

There is a significant need for novel methods for treating or lessening the severity of cystic fibrosis in a patient. The present invention satisfies these needs.

### SUMMARY

The present invention relates to methods for treating patients with CF, an autosomal recessive hereditary disease caused by a mutation in the gene CFTR. Mutations in the CFTR protein result in loss of CFTR activity at the surface of epithelial cells leading to abnormal ion transport, dehydration of secretions, mucosal obstruction of exocrine glands, and an altered inflammatory response, especially in the lungs. Multiple organ systems are involved, most notably the respiratory and gastrointestinal (GI) systems. Pulmonary problems are characterized by airway obstruction, impaired mucociliary clearance, inflammation, and infection. The ultimate goal of CFTR modulator therapy is to maximize and maintain CFTR function, thereby restoring chloride transport. CF is diagnosed by the levels of chloride in sweat and people with CF have elevated sweat chloride levels. Interventions that modulate CFTR activity use sweat chloride as a clinical biomarker of effect. Reduction in sweat chloride levels indicates direct modulation of CFTR.

The present invention provides methods for treating or lessening the severity of CF, comprising the step of administering to a patient in need an effective amount of an S-nitrosoglutathione reductase (“GSNOR”) inhibitor in combination with one or more secondary active agents. The GSNOR inhibitor of the method can be administered concurrently with, prior to, or subsequent to, one or more secondary active agents. The invention encompasses pharmaceutically acceptable salts, stereoisomers, prodrugs, metabolites, and N-oxides of the described compounds. Also encompassed by the invention are pharmaceutical compositions comprising at least one GSNOR inhibitor and at least one pharmaceutically acceptable carrier. Also encompassed by the invention are pharmaceutical compositions comprising at least one GSNOR inhibitor for the treatment of cystic fibrosis. Also encompassed by the invention are pharmaceutical compositions comprising a GSNOR inhibitor in combination with one or more secondary active agents. Also encompassed by the invention are pharmaceutical compositions comprising a GSNOR inhibitor in combination with one or more secondary active agents for the treatment of cystic fibrosis.

Also encompassed by the invention are methods that include administering a therapeutically effective amount of a GSNOR inhibitor for the treatment of cystic fibrosis administered with one or more secondary active agents, and wherein the combination can be administered with one or more palliative agents.

The compositions of the present invention can be prepared in any suitable pharmaceutically acceptable dosage form.

Although methods and materials similar or equivalent to those described herein can be used in the practice or testing of the present invention, suitable methods and materials are described below. All publicly available publications, patent applications, patents, and other references mentioned herein are incorporated by reference in their entirety. In the case of conflict, the present specification, including definitions, will control.

Both the foregoing summary and the following detailed description are exemplary and explanatory and are intended to provide further details of the compositions and methods as claimed. Other objects, advantages, and novel features will be readily apparent to those skilled in the art from the following detailed description.

### BRIEF DESCRIPTION OF THE FIGURES

FIGS. 1A and 1B: CFTR-mediated Short Circuit current measurements. FIG. 1A: Well-differentiated CF (F508del/F508del) HBE cells were treated with vehicle alone (DMSO), CFTR corrector^{††} (3-{6-{[1-(2,2-difluoro-1,3-benzodioxol-5-yl) cyclopropanecarbonyl]amino}-3-methylpyridin-2-yl}benzoic acid), or a combination of CFTR corrector^{††} and GSNORi* (3-chloro-4-(6-hydroxyquinolin-2-yl)benzoic acid). Representative short circuit current (Isc) recordings are shown. FIG. 1B: Summary of the overall change in Isc (ΔIsc) for total CFTR stimulation as well as CFTR channel inhibition are shown.

FIGS. 2A, 2B, and 2C: Modulation of F508del-CFTR-mediated ion transport with combination treatment using primary human airway epithelial cells. Well-differentiated CF (F508del/F508del) HAE cells were mounted into Using chambers and treated with vehicle alone (DMSO), CFTR corrector^{††} (3-{6-{[1-(2,2-difluoro-1,3-benzodioxol-5-yl)cyclopropanecarbonyl]amino}-3-methylpyridin-2-yl}benzoic acid)+CFTR potentiator^{†} N-(2,4-Di-tert-butyl-5-hydroxyphenyl)-4-oxo-1,4-dihydroquinoline-3-carboxamide), or CFTR corrector^{††} +CFTR potentiator^{†}+GSNORi* (3-chloro-4-(6-hydroxyquinolin-2-yl)benzoic acid). FIG. 2A shows short circuit current (I_{sc}) recordings for all 3 conditions. In FIG. 2B, the slope and in FIG. 2C, the area under the curve (AUC) for CFTR-stimulated I_{sc} was quantitated.

FIGS. 3A and 3B: Using Chamber experiments. FIG. 3A shows the slope measurements calculated from a time point immediately after the maximal forskolin Isc is achieved to a time point immediately before the addition of 172. FIG. 3B depicts the area under the curve (AUC) which was quantitated as the net Cl⁻ secretion measured from a time point immediately before the addition of forskolin and a horizontal extension to a time point after the maximal 172 inhibition has been achieved.

FIG. 4A and FIG. 4B: Slope Fold Change and AUC. Data from experiments of FIGS. 3A and 3B is transposed into fold change by dividing the value obtained from VX-809+VX-770+3-chloro-4-(6-hydroxyquinolin-2-yl)benzoic acid treatment by the value obtained by VX-809 and VX-770 treatment alone. FIG. 4A shows the fold change in slope and FIG. 4B shows the fold change AUC.

FIG. 5A shows representative short-circuit current (I_{SC}) tracings from CF (F508del/F508del) HBE cells obtained from two patient codes. FIG. 5B shows the magnitude of the I_{SC} that is inhibited by 172 of CF cells exposed to VX-809+GSNORi* versus VX-809 alone.

FIG. 6: AUC is measured as described in Example 1 is shown for 3-chloro-4-(6-hydroxyquinolin-2-yl) benzoic acid plus VX-809 compared to VX-809 alone. Data is with 2 patient codes, 6 inserts.

FIGS. 7A and 7B: FIGS. 7A and 7B represent—Using chamber analyses where AUC was measured. FIG. 7A: VX-809 versus VX-809+3-chloro-4-(6-hydroxyquinolin-2-yl)benzoic acid. FIG. 7B: VX-809+VX-770 versus VX-809/VX-770+3-chloro-4-(6-hydroxyquinolin-2-yl)benzoic acid).

FIGS. 8A and 8B: Immunoprecipitation (IP) and Western blot analysis to detect both Band B (immature) and Band C (mature) FIG. 8A: Western Blot and FIG. 8B: histobars show an increase in both CFTR band B and C are detected when cells are treated with the combination of VX-809+3-chloro-4-(6-hydroxyquinolin-2-yl) benzoic acid compared to VX-809 alone.

FIG. 9: Immunoprecipitation/Western Blot Analysis using primary CF (F508del-CFTR) HAE cells.

FIGS. 10A, 10B, and 10C: Well-differentiated CF (F508del/F508del) HAE cells were mounted into Using chambers (KBR/KBR) and treated with, DMSO control, VX-809 or VX-809+GSNORi* (3-chloro-4-(6-hydroxyquinolin-2-yl) benzoic acid). FIG. 10A: A representative trace showing short circuit current (I_{sc}) is shown. FIG. 10B: The area under the curve (AUC) for total CFTR-stimulated I_{sc} was quantitated for cells treated with VX-809 alone or VX-809+GSNORi. FIG. 10C: the AUC fold change is shown.

FIGS. 11A, 11B, 11C and 11D: Well-differentiated CF (F508del/F508del) HAE cells were mounted into Using chambers (KBR/KBR) and treated with, DMSO control, VX-809+VX-770 or VX-809+VX-770+GSNORi* (3-chloro-4-(6-hydroxyquinolin-2-yl) benzoic acid). FIG. 11A: A representative trace showing short circuit current (I_{sc}) is shown. FIG. 11B: The area under the curve (AUC) for total CFTR-stimulated Is, was quantitated for cells treated with VX-809+VX-770 and VX-809+VX770+GSNORi*. FIG. 11C: Fold change for AUC was quantitated such to normalize within different CF patient derived cells. FIG. 11D: The fold change was determined for the slope of total CFTR-stimulated I_{sc}.

FIG. 12: Well-differentiated CF (F508del/F508del) HAE cells were treated with either VX-809 or VX-809+GSNORi* (3-chloro-4-(6-hydroxyquinolin-2-yl) benzoic acid). I_{sc} was monitored. Using Chamber ΔIsc (fold change) in response to 3 μM vx-770 (apical) and 100 μM CFTRinh172 (apical) are shown in FIG. 12.

FIGS. 13A and 13B: Western Blot analysis detecting CFTR B-band (immature) and C-band (mature). Western blot gel (FIG. 13A) and histobars (FIG. 13B) represent changes in CFTR protein expression (C-band/B-band ratio) using FRT (F508del-CFTR) cells treated with 3-chloro-4-(6-hydroxyquinolin-2-yl) benzoic acid (GSNORi*), VX-809, and the combination of VX-809+3-chloro-4-(6-hydroxyquinolin-2-yl) benzoic acid (GSNORi*).

FIG. 14: Summary table representing changes in CFTR protein expression. Western blot analysis quantitating the ratio of CFTR C-band/B-band (fold change) and % increase is shown from FRT (F508del-CFTR) cells treated with the combination of VX-809+GSNORi* (3-chloro-4-(6-hydroxyquinolin-2-yl) benzoic acid) compared to VX-809 alone.

FIGS. 15A and 15B: Effect of GSNORi* on the plasma membrane (PM) density of F508del-CFTR expressed in the human bronchial cell line CFBE41o- (CFBE). FIG. 15A: The bar graph shows the effect of 100 μM, 24 h GSNORi* in combination with DMSO, VX-809, or VX-661. FIG. 15B: CFBE cells were treated with GSNORi* (100 μM, 24 h) or DMSO alone or in combination with VX-809 (3 μM, 24 h), VX-809+VX-770 (1 μM, 24 h), VX-661 (3 μM, 24 h), or VX-661+VX-770. The plasma membrane densities were determined by cell surface ELISA.

### DETAILED DESCRIPTION

### A. Overview of the Invention

In accord with the present invention, GSNOR has been shown to function in vivo and in vitro to metabolize S-nitrosoglutathione (GSNO) and protein S-nitrosothiols (SNOs) to modulate NO bioactivity, by controlling the intracellular levels of low mass NO donor compounds and preventing protein nitrosylation from reaching toxic levels.

Based on this, it follows that inhibition of this enzyme potentiates bioactivity in diseases in which NO donor therapy is indicated, inhibits the proliferation of pathologically proliferating cells, and increases NO bioactivity in diseases where this is beneficial.

Cystic fibrosis (CF) is a lethal genetic disease affecting 70,000 people worldwide. Approximately one in 3,500 children in the US is born with CF each year. It is a disease that affects all racial and ethnic groups, but is more common among Caucasians. An estimated 30,000 American adults and children have CF, and the median predicted age of survival is 37.4 years (CFF Registry Report 2007, Cystic Fibrosis Foundation, Bethesda, Md.). CF is an autosomal recessive hereditary disease caused by a mutation in the gene for the cystic fibrosis transmembrane regulator (CFTR) protein. CFTR aids the regulation of epithelial salt and water transport in multiple organs, including the lung, pancreas, liver, and intestinal tract. Clinical manifestations of CF include abnormal sweat electrolytes, chronic and progressive respiratory disease, exocrine pancreatic dysfunction, and infertility; however, it is lung disease that is the primary cause of morbidity and mortality. In the lung, the loss of CFTR mediated Cl⁻ secretion is believed to cause airway surface dehydration due to both a decrease in CFTR-mediated Cl and fluid secretion and a secondary increase in epithelial Na⁺ channel (ENaC)-mediated Na⁺ and fluid absorption. This imbalance results in dehydration of the airway surface, and likely contributes to the deleterious cascade of mucus accumulation, infection, inflammation, and destruction that characterizes CF lung disease. The accumulation of mucus leads to plugging in the passageways in the lung and other organs, such as the pancreas.

Current therapies to treat CF lung disease, including mucolytics, antibiotics, anti-inflammatory agents, anti-infectives and nutritional agents, target the downstream disease consequences that are secondary to the loss of CFTR function. Since the median predicted survival age is currently about 37 years, there is a large medical need for more efficacious therapies that address the underlying defect of CF.

To address this need, there has been increased interest in small-molecule therapies that increase CFTR function because such an approach could address the consequences of CFTR dysfunction as well as slow the progression of the disease. Such therapies are broadly classified as CFTR modulators and include CFTR activators, potentiators, correctors, and antagonists. CFTR activators act on their own to stimulate CFTR-mediated ion transport and include agents that increase cAMP levels, such as b-adrenergic agonists, adenylate cyclase activators, and phosphodiesterase inhibitors. CFTR potentiators act in the presence of endogenous or pharmacological CFTR activators to increase the channel gating activity of cell-surface localized CFTR, resulting in enhanced ion transport. CFTR correctors act by increasing the delivery and amount of functional CFTR protein to the cell surface, resulting in enhanced ion transport. Depending on the molecular consequence of the mutation and disease severity. CFTR activators, potentiators, and correctors may be coadministered to maximize clinical efficacy or therapeutic window, if needed. CFTR antagonists act by decreasing CFTR-mediated ion transport and are being developed for the treatment of polycystic kidney disease and cholera-induced secretory diarrhea.

There are many (>1500) different gene mutations for CF. Mutations affecting the CFTR gene cause a large variety of defects including altered CFTR channel gating (class III mutations such as G551D and G1349D) or impaired CFTR protein maturation (class II mutations such as F508del). Therefore, compounds increasing CFTR-dependent chloride transport are potentially useful as drugs to treat CF patients. In particular, pharmacological activators of CFTR, called potentiators, are useful to overcome the gating defect caused by class III CF mutations. Conversely, other compounds, called correctors, may help the F508del-CFTR protein to escape the endoplasmic reticulum and reach the plasma membrane. Potentiators are also useful for F508del. Indeed, this mutation causes also a gating defect, although less severe than that of classical class III mutations. On the other hand, CFTR inhibitors are characterized by decreased CFTR activity.

The most common mutation, F508del-CFTR (class II), results from a 3 base pair deletion that leads to the deletion of phenylalanine at position 508 of the full-length protein. The resulting F508del-CFTR protein is unstable and susceptible to rapid degradation in the 26S proteosome, with little if any F508del-CFTR at the plasma membrane. The F508del mutation is found in approximately 86% of all CF patients in the United States and Europe. In the F508del mutation, the deletion of an amino acid results in misfolded CFTR that is unstable and is targeted for degradation, which is facilitated by chaperone proteins. As a result, not enough CFTR reaches, or “traffics” to, the cell surface. F508del is therefore referred to as a trafficking mutation. In the United States, approximately 47% of CF patients are homozygous and have two copies of this mutation, and approximately 39% are heterozygous and have one copy.

In the lungs of CF patients, the lack of transport of chloride and accompanying water across the airway epithelium and excessive sodium reabsorption leads to dehydrated airway surface fluid, impaired mucociliary clearance, infection and inflammation. Increasing the amount of F508-CFTR that reaches the plasma membrane, or otherwise improving its function, offers the potential to improve the hydration of the airway surface fluid and reverse part of the underlying pathophysiology.

Inhibitors of S-nitrosoglutathione reductase (GSNOR), the primary catabolizing enzyme of S-nitrosoglutathione (GSNO), may provide a novel therapeutic strategy in cystic fibrosis (CF). GSNO has been identified as a potential modulator of CFTR; however, attempts to deliver GSNO exogenously are fraught with difficulties related to formulation, intracellular delivery, and inconsistency of results. GSNOR inhibitors on the other hand are distinguished by their ability to consistently demonstrate preservation of intracellular GSNO and potent bronchodilatory and anti-inflammatory effects in animal models of COPD and asthma.

Increasing the amount of F508-CFTR that reaches the plasma membrane, or otherwise improving its function, offers the potential to improve the hydration of the airway surface fluid and reverse part of the underlying pathophysiology. It is believed that GSNOR inhibition can increase CFTR mediated chloride transport. Mechanisms by which GSNOR inhibitors may improve F508del-CFTR function include nitrosation of chaperone proteins potentially improving the stability of the misfolded protein allowing it to move beyond a stalled folding intermediate(s) (Coppinger et al., 2012), prevention of CFTR proteosomal degradation, promotion of CFTR maturation, and maintenance of epithelial tight junctions.

The potential benefits of GSNOR inhibitors in CF extend beyond their potential to affect chloride and water transport and to increase the airway surface fluid level. They may also affect what appears to be a primary defect in local mucosal immunity. Cohen and Prince have noted that even in the absence of clinically apparent viral or bacterial infection, there is often evidence of inflammation in CF airways, as evidenced by polymorphonuclear neutrophil (PMN) accumulation and excessive concentrations of interleukin-8 (IL-8) and free proteases, accompanied by over-activated nuclear factor kappa B (NFκB) and ineffective antioxidant transport (Cohen and Prince, 2012).

The anti-inflammatory properties of GSNOR inhibition have been demonstrated in several in vitro and in vivo models. Of particular relevance to cystic fibrosis are the mouse models of COPD (cigarette smoke and elastase/papain) in which cellular influx was prevented or reversed, and epithelial cell damage was minimized. The relevance of these models to CF lung disease lies in their common inflammatory manifestations of NFκB activation, neutrophilic infiltration, and elastase-mediated lung injury. GSNOR inhibition has been shown to down regulate the activity of transcription factor NFκB by nitrosation of NFκB regulatory proteins. GSNOR inhibition, therefore, offers a novel mechanism for targeting inflammatory pathways in CF.

The combination of improved F508del-CFTR and anti-inflammatory effects arising from GSNOR inhibition may lead to clinical improvement in CF patients, which may be preceded by measurable changes in FEV₁, sweat chloride, NPD, and inflammatory biomarkers in serum and airway secretions, sputum and/or bronchoalveolar lavage fluid (BALF). Other measurements of clinical improvement may be intestinal current measurements and weight gain.

The present invention provides methods and pharmaceutical compositions that are useful in treating or lessening the severity of cystic fibrosis in a patient by administering to said patient an effective amount of a GSNOR inhibitor in combination with one or more secondary active agents. The GSNOR inhibitor of the pharmaceutical composition can be administered concurrently with, prior to, or subsequent to, one or more secondary active agents.

In one embodiment, the present invention provides methods of treating or lessening the severity of cystic fibrosis in a patient by administering to said patient a therapeutically effective amount of
i) a GSNOR inhibitor or a pharmaceutically acceptable salt thereof, in combination with
ii) one or more secondary active agents selected from the group consisting of CFTR correctors and CFTR potentiators or pharmaceutically acceptable salt(s) thereof.

The methods of the present invention include the administration of the pharmaceutical compositions as described herein.

In one embodiment, the present invention provides a pharmaceutical composition, comprising
i) a GSNOR inhibitor or a pharmaceutically acceptable salt thereof, in combination with
ii) one or more secondary active agents selected from the group consisting of CFTR correctors and CFTR potentiators or pharmaceutically acceptable salt(s) thereof.

In one of its aspects, the present invention provides a pharmaceutical composition as described above wherein the GSNOR inhibitor is selected from a compound with the formula I or a pharmaceutically acceptable salt thereof:

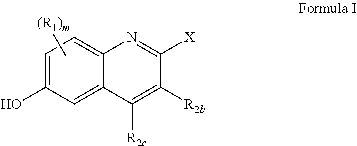

wherein
m is selected from the group consisting of 0, 1, 2, or 3;
R₁ is independently selected from the group consisting of chloro, fluoro, bromo, cyano, and methoxy; R_{2b} and R_{2c} are independently selected from the group consisting of hydrogen, halogen, C₁-C₃ alkyl, fluorinated C₁-C₃ alkyl, cyano, C₁-C₃ alkoxy, and N(CH₃)₂;
X is selected from the group consisting of

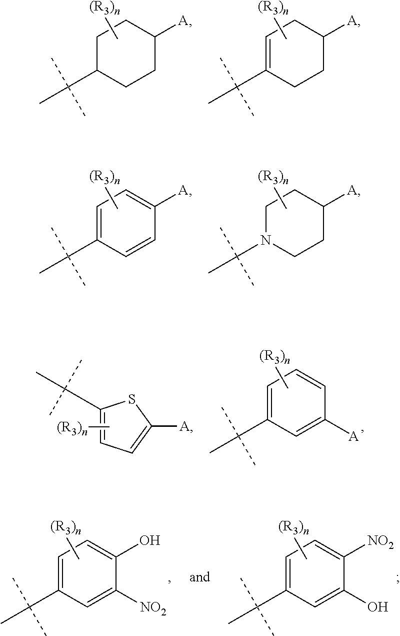

n is selected from the group consisting of 0, 1, and 2;
R₃ is independently selected from the group consisting of halogen, C₁-C₃ alkyl, fluorinated C₁-C₃ alkyl, cyano, C₁-C₃ alkoxy, and NR₄R_{4′} where R₄ and R_{4′} are independently selected from the group consisting of C₁-C₃ alkyl, or R₄ when taken together with R_{4′} form a ring with 3 to 6 members; and
A is selected from the group consisting of

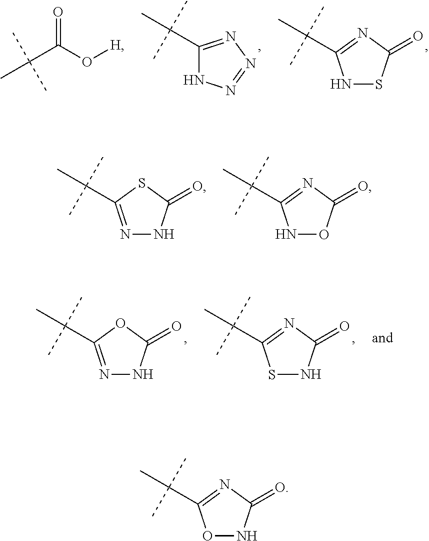

In one embodiment, the GSNOR inhibitor of the pharmaceutical composition is a compound of Formula I wherein R₁ is independently selected from the group consisting of chloro, fluoro, and bromo; R₃ is independently selected from the group consisting of halogen, C₁-C₃ alkyl, fluorinated C₁-C₃ alkyl, cyano, C₁-C₃ alkoxy, and NR₄R_{4′} where R₄ and R_{4′} are independently selected from the group consisting of C₁-C₃ alkyl, or R₄ when taken together with R_{4′} form a ring with 3 to 6 members; and

X is selected from the group consisting of

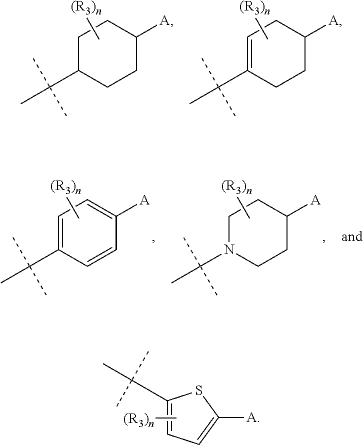

In one embodiment, the GSNOR inhibitor of the pharmaceutical composition is a compound of Formula I wherein R₃ is independently selected from the group consisting of halogen, C₁-C₃ alkyl, fluorinated C₁-C₃ alkyl, cyano, C₁-C₃ alkoxy, and NR₄R_{4′} where R₄ and R_{4′} are methyl, or alternatively together with the said N form the ring aziridin-1-yl or morpholino.

In one embodiment, the GSNOR inhibitor of the pharmaceutical composition is a compound of Formula I wherein m is selected from the group consisting of 0 and 1; R_{2b} and R_{2c} are independently selected from the group consisting of hydrogen, chloro, fluoro, methyl, trifluoromethyl, cyano, methoxy, and N(CH₃)₂; n is selected from the group consisting of 0 and 1; and R₃ is independently selected from the group consisting of fluoro, chloro, bromo, methyl, trifluoromethyl, cyano, hydroxy, methoxy, and N(CH₃)₂.

In one embodiment, the GSNOR inhibitor of the pharmaceutical composition is a compound of Formula I wherein X is

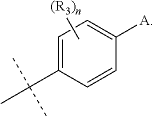

In one embodiment, the GSNOR inhibitor of the pharmaceutical composition is a compound of Formula I wherein A is COOH.

In one embodiment, the GSNOR inhibitor of the pharmaceutical composition is a compound of Formula I wherein the compound of Formula I is selected from:
4-(6-hydroxy-3-methylquinolin-2-yl)benzoic acid;
2-(4-(1H-tetrazol-5-yl)phenyl)-3-methylquinolin-6-ol;
4-(6-hydroxyquinolin-2-yl)benzoic acid;
2-(4-(1H-tetrazol-5-yl)phenyl)quinolin-6-ol;
1-(6-hydroxyquinolin-2-yl)piperidine-4-carboxylic acid;
(1r,4r)-4-(6-hydroxyquinolin-2-yl)cyclohexanecarboxylic acid;
(1s,4s)-4-(6-hydroxyquinolin-2-yl)cyclohexanecarboxylic acid;
3-chloro-4-(6-hydroxyquinolin-2-yl)benzoic acid;
2-chloro-4-(6-hydroxyquinolin-2-yl)benzoic acid;
2-fluoro-4-(6-hydroxyquinolin-2-yl)benzoic acid;
2-(4-(2H-tetrazol-5-yl)phenyl)-4-chloroquinolin-6-ol;
3-(4-(6-hydroxyquinolin-2-yl)phenyl)-1,2,4-oxadiazol-5 (2H)-one;
3-fluoro-4-(6-hydroxyquinolin-2-yl)benzoic acid;
4-(6-hydroxyquinolin-2-yl)-3-methoxybenzoic acid;
5-(6-hydroxyquinolin-2-yl)thiophene-2-carboxylic acid;
4-(6-hydroxyquinolin-2-yl)cyclohex-3-enecarboxylic acid;
4-(3-fluoro-6-hydroxyquinolin-2-yl)benzoic acid;
4-(4-chloro-3-fluoro-6-hydroxyquinolin-2-yl)benzoic acid;
4-(3-chloro-6-hydroxyquinolin-2-yl)benzoic acid;
3-(2-fluoro-4-(6-hydroxyquinolin-2-yl)phenyl)-1,2,4-oxadiazol-5 (4H)-one;
3-(3-fluoro-4-(6-hydroxyquinolin-2-yl)phenyl)-1,2,4-oxadiazol-5 (4H)-one;
4-(4-chloro-6-hydroxyquinolin-2-yl)benzoic acid;
2-(2-chloro-4-(2H-tetrazol-5-yl)phenyl)quinolin-6-ol;
5-(4-(6-hydroxyquinolin-2-yl)phenyl)-1,3,4-oxadiazol-2(3H)-one;
3-(dimethylamino)-4-(6-hydroxyquinolin-2-yl)benzoic acid;
4-(4-fluoro-6-hydroxyquinolin-2-yl)benzoic acid;
4-(6-hydroxyquinolin-2-yl)-3-methylbenzoic acid;
4-(3-chloro-6-hydroxyquinolin-2-yl)-3-fluorobenzoic acid;
3-(4-(6-hydroxyquinolin-2-yl)phenyl)-1,2,4-thiadiazol-5 (2H)-one;
4-(6-hydroxyquinolin-2-yl)-3-(trifluoromethyl)benzoic acid;
4-(6-hydroxy-3-(trifluoromethyl)quinolin-2-yl)benzoic acid;
2-(4-carboxyphenyl)-6-hydroxyquinoline 1-oxide;
5-(4-(6-hydroxyquinolin-2-yl)phenyl)-1,3,4-thiadiazol-2(3H)-one;
5-(4-(6-hydroxyquinolin-2-yl)phenyl)-1,2,4-oxadiazol-3 (2H)-one;
(1r,4r)-4-(3-chloro-6-hydroxyquinolin-2-yl)cyclohexanecarboxylic acid;
(1s,4s)-4-(3-chloro-6-hydroxyquinolin-2-yl)cyclohexanecarboxylic acid;
3-chloro-4-(4-fluoro-6-hydroxyquinolin-2-yl)benzoic acid;
2-(5-(2H-tetrazol-5-yl)thiophen-2-yl)quinolin-6-ol;
5-(4-(6-hydroxyquinolin-2-yl)phenyl)-1,2,4-thiadiazol-3 (2H)-one;
3-fluoro-4-(4-fluoro-6-hydroxyquinolin-2-yl)benzoic acid;
1-(6-hydroxy-3-(trifluoromethyl)quinolin-2-yl)piperidine-4-carboxylic acid;
4-(5-chloro-6-hydroxyquinolin-2-yl)benzoic acid;
(1r,4r)-4-(6-hydroxy-3-(trifluoromethyl)quinolin-2-yl)cyclohexanecarboxylic acid;
(1s,4s)-4-(6-hydroxy-3-(trifluoromethyl)quinolin-2-yl)cyclohexanecarboxylic acid;
4-(5-bromo-6-hydroxyquinolin-2-yl)benzoic acid;
3-bromo-4-(6-hydroxyquinolin-2-yl)benzoic acid;
4-(4-(dimethylamino)-6-hydroxyquinolin-2-yl)benzoic acid;
4-(4-fluoro-6-hydroxyquinolin-2-yl)-3-methoxybenzoic acid;
3-cyano-4-(6-hydroxyquinolin-2-yl)benzoic acid;
2-(4-carboxy-2-chlorophenyl)-6-hydroxyquinoline 1-oxide;
4-(3-cyano-6-hydroxyquinolin-2-yl)benzoic acid;
4-(5-fluoro-6-hydroxyquinolin-2-yl)benzoic acid;
4-(8-fluoro-6-hydroxyquinolin-2-yl)benzoic acid;
and
3-fluoro-4-(5-fluoro-6-hydroxyquinolin-2-yl)benzoic acid.

In one embodiment, the GSNOR inhibitor of the pharmaceutical composition is 3-chloro-4-(6-hydroxyquinolin-2-yl)benzoic acid.

In one embodiment, the GSNOR inhibitor of the pharmaceutical composition is 3-fluoro-4-(6-hydroxyquinolin-2-yl)benzoic acid.

In one embodiment, the GSNOR inhibitor of the pharmaceutical composition is 4-(6-hydroxyquinolin-2-yl)-3-methylbenzoic acid.

In one embodiment, the invention provides a pharmaceutical composition as described previously wherein the secondary active agent(s) are selected from CFTR correctors and CFTR potentiators.

In one embodiment, the secondary active agent of the pharmaceutical composition is a CFTR corrector.

In one embodiment, the secondary active agent of the pharmaceutical composition is a CFTR potentiator.

In one embodiment, a secondary active agent of the pharmaceutical composition is a CFTR corrector. In one embodiment, the CFTR corrector is selected from the group consisting of 3-{6-{[1-(2,2-difluoro-1,3-benzodioxol-5-yl)cyclopropanecarbonyl]amino}-3-methylpyridin-2-yl}benzoic acid, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, and 5-{6-[2-(2,2-difluoro-2H-1,3-benzodioxol-5-yl)-2-methylpropanamido]-3-methylpyridin-2-yl}thiophene-3-carboxylic acid.

In one embodiment, a secondary active agent of the pharmaceutical composition is a CFTR potentiator. In one embodiment, the CFTR potentiator is N-(2,4-Di-tert-butyl-5-hydroxyphenyl)-4-oxo-1,4-dihydroquinoline-3-carboxamide.

In one embodiment, the pharmaceutical composition comprises a GSNOR inhibitor selected from the group consisting of 3-chloro-4-(6-hydroxyquinolin-2-yl)benzoic acid, 3-fluoro-4-(6-hydroxyquinolin-2-yl)benzoic acid, and 4-(6-hydroxyquinolin-2-yl)-3-methylbenzoic acid in combination with the CFTR potentiator is N-(2,4-Di-tert-butyl-5-hydroxyphenyl)-4-oxo-1,4-dihydroquinoline-3-carboxamide.

In one embodiment, the pharmaceutical composition comprises a GSNOR inhibitor selected from the group consisting of 3-chloro-4-(6-hydroxyquinolin-2-yl)benzoic acid, 3-fluoro-4-(6-hydroxyquinolin-2-yl)benzoic acid, and 4-(6-hydroxyquinolin-2-yl)-3-methylbenzoic acid in combination with a CFTR corrector selected from the group consisting of of 3-{6-{[1-(2,2-difluoro-1,3-benzodioxol-5-yl)cyclopropanecarbonyl]amino}-3-methylpyridin-2-yl}benzoic acid, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, and 5-{6-[2-(2,2-difluoro-2H-1,3-benzodioxol-5-yl)-2-methylpropanamido]-3-methylpyridin-2-yl}thiophene-3-carboxylic acid.

In one embodiment, the secondary active agents of the pharmaceutical composition are a CFTR corrector and a CFTR potentiator.

In one embodiment, the pharmaceutical composition includes two secondary active agents wherein the first is the CFTR potentiator N-(2,4-Di-tert-butyl-5-hydroxyphenyl)-4-oxo-1,4-dihydroquinoline-3-carboxamide, and the second is a CFTR corrector selected from the group consisting of 3-{6-{[1-(2,2-difluoro-1,3-benzodioxol-5-yl)cyclopropanecarbonyl]amino}-3-methylpyridin-2-yl}benzoic acid, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, and 5-{6-[2-(2,2-difluoro-2H-1,3-benzodioxol-5-yl)-2-methylpropanamido]-3-methylpyridin-2-yl}thiophene-3-carboxylic acid.

In one embodiment, the pharmaceutical composition comprises the GSNOR inhibitor 3-chloro-4-(6-hydroxyquinolin-2-yl)benzoic acid in combination with two secondary active agents wherein the first is N-(2,4-Di-tert-butyl-5-hydroxyphenyl)-4-oxo-1,4-dihydroquinoline-3-carboxamide and the second is 3-{6-{[1-(2,2-difluoro-1,3-benzodioxol-5-yl)cyclopropanecarbonyl]amino}-3-methylpyridin-2-yl}benzoic acid.

In one embodiment, the pharmaceutical composition comprises the GSNOR inhibitor 3-fluoro-4-(6-hydroxyquinolin-2-yl)benzoic acid in combination with two secondary active agents wherein the first is N-(2,4-Di-tert-butyl-5-hydroxyphenyl)-4-oxo-1,4-dihydroquinoline-3-carboxamide and the second is 3-{6-{[1-(2,2-difluoro-1,3-benzodioxol-5-yl)cyclopropanecarbonyl]amino}-3-methylpyridin-2-yl}benzoic acid.

In one embodiment, the pharmaceutical composition comprises the GSNOR inhibitor 4-(6-hydroxyquinolin-2-yl)-3-methylbenzoic acid in combination with two secondary active agents wherein the first is N-(2,4-Di-tert-butyl-5-hydroxyphenyl)-4-oxo-1,4-dihydroquinoline-3-carboxamide and the second is 3-{6-{[1-(2,2-difluoro-1,3-benzodioxol-5-yl)cyclopropanecarbonyl]amino}-3-methylpyridin-2-yl}benzoic acid.

In one embodiment, the pharmaceutical composition comprises the GSNOR inhibitor 3-chloro-4-(6-hydroxyquinolin-2-yl)benzoic acid in combination with two secondary active agents wherein the first is N-(2,4-Di-tert-butyl-5-hydroxyphenyl)-4-oxo-1,4-dihydroquinoline-3-carboxamide and the second is 5-{6-[2-(2,2-difluoro-2H-1,3-benzodioxol-5-yl)-2-methylpropanamido]-3-methylpyridin-2-yl}thiophene-3-carboxylic acid.

In one embodiment, the pharmaceutical composition comprises the GSNOR inhibitor 3-fluoro-4-(6-hydroxyquinolin-2-yl)benzoic acid in combination with two secondary active agents wherein the first is N-(2,4-Di-tert-butyl-5-hydroxyphenyl)-4-oxo-1,4-dihydroquinoline-3-carboxamide and the second is 5-{6-[2-(2,2-difluoro-2H-1,3-benzodioxol-5-yl)-2-methylpropanamido]-3-methylpyridin-2-yl}thiophene-3-carboxylic acid.

In one embodiment, the pharmaceutical composition comprises the GSNOR inhibitor 4-(6-hydroxyquinolin-2-yl)-3-methylbenzoic acid in combination with two secondary active agents wherein the first is N-(2,4-Di-tert-butyl-5-hydroxyphenyl)-4-oxo-1,4-dihydroquinoline-3-carboxamide and the second is 5-{6-[2-(2,2-difluoro-2H-1,3-benzodioxol-5-yl)-2-methylpropanamido]-3-methylpyridin-2-yl}thiophene-3-carboxylic acid.

In one embodiment, the secondary active agent of the composition is selected from the following
3-{6-[2-(3-chlorophenyl)-2-methylpropanamido]-3-methylpyridin-2-yl}benzoic acid;
3-{6-[2-(3,4-dichlorophenyl)-2-methylpropanamido]-3-methylpyridin-2-yl}benzoic acid;
3-{6-[2-(3-fluorophenyl)-2-methylpropanamido]-3-methylpyridin-2-yl}benzoic acid;
3-{6-[2-(3,4-difluorophenyl)-2-methylpropanamido]-3-methylpyridin-2-yl}benzoic acid;
3-{6-[2-(4-chlorophenyl)-2-methylpropanamido]-3-methylpyridin-2-yl}benzoic acid;
3-{6-[2-(2,2-difluoro-2H-1,3-benzodioxol-5-yl)propanamido]-3-methylpyridin-2-yl}benzoic acid;
3-{6-[2-(2,2-difluoro-2H-1,3-benzodioxol-5-yl)-2-methylpropanamido]-3-methylpyridin-2-yl}benzoic acid;
5-{6-[2-(2,2-difluoro-2H-1,3-benzodioxol-5-yl)-2-methylpropanamido]-3-methylpyridin-2-yl}thiophene-3-carboxylic acid;
5-{6-[2-(2,2-difluoro-2H-1,3-benzodioxol-5-yl)-2-methylbutanamido]-3-methylpyridin-2-yl}thiophene-3-carboxylic acid;
3-{6-[2-(4-methoxyphenyl)-2-methylpropanamido]-3-methylpyridin-2-yl}benzoic acid;
3-(3-methyl-6-{2-methyl-2-[4-(trifluoromethyl)phenyl]propanamido}pyridine-2-yl)benzoic acid;
3-(3-methyl-6-{2-methyl-2-[3-(trifluoromethyl)phenyl]propanamido}yridine-2-yl)benzoic acid;
3-{6-[2-(3-methoxyphenyl)-2-methylpropanamido]-3-methylpyridin-2-yl}benzoic acid;
2-(2,2-difluoro-2H-1,3-benzodioxol-5-yl)-N-{6-[3-(hydroxymethyl)phenyl]-5-methylpyridin-2-yl}-2-methylpropanamide;
3-{6-[2-(2,2-difluoro-2H-1,3-benzodioxol-5-yl)-3-hydroxy-2-methylpropanamido]-3-methylpyridin-2-yl}benzoic acid;
2-(2,2-difluoro-2H-1,3-benzodioxol-5-yl)-N-[6-(3-hydroxypiperidin-1-yl)-5-methylpyridin-2-yl]-2-methylpropanamide;
1-{6-[2-(2,2-difluoro-2H-1,3-benzodioxol-5-yl)-2-methylpropanamido]-3-methylpyridin-2-yl}piperidine-3-carboxylic acid;
2-(2,2-difluoro-2H-1,3-benzodioxol-5-yl)-N-{6-[3-(hydroxymethyl)piperidin-1-yl]-5-methylpyridin-2-yl}-2-methylpropanamide;
5-{6-[2-(2,2-difluoro-2H-1,3-benzodioxol-5-yl)-2-methylpropanamido]-3-methylpyridin-2-yl}thiophene-2-carboxylic acid;
5-{6-[2-(2,2-difluoro-2H-1,3-benzodioxol-5-yl)-2-methylpropanamido]-3-methylpyridin-2-yl}-2-fluorobenzoic acid;
4-{6-[2-(2,2-difluoro-2H-1,3-benzodioxol-5-yl)-2-methylpropanamido]-3-methylpyridin-2-yl}thiophene-2-carboxylic acid;
3-{6-[2-cyano-2-(2,2-difluoro-2H-1,3-benzodioxol-5-yl)-2-methylacetamido]-3-methylpyridin-2-yl}benzoic acid;
3-{6-[2-(2,2-difluoro-2H-1,3-benzodioxol-5-yl)-2-methylpropanamido]-3-methylpyridin-2-yl}-4-fluorobenzoic acid;
3-{6-[2-(2,2-difluoro-2H-1,3-benzodioxol-5-yl)-2-methylpropanamido]-3-methylpyridin-2-yl}-5-fluorobenzoic acid;
3-{6-[2-(2,2-difluoro-2H-1,3-benzodioxol-5-yl)-2-methylpropanamido]-3-methylpyridin-2-yl}-2-fluorobenzoic acid;
3-{6-[2-(2,2-difluoro-2H-1,3-benzodioxol-5-yl)-2-methylpropanamido]-3-fluoropyridin-2-yl}benzoic acid;
2-(2,2-difluoro-2H-1,3-benzodioxol-5-yl)-N-[6-(3-hydroxyphenyl)-5-methylpyridin-2-yl]-2-methylpropanamide;
2-(2,2-difluoro-2H-1,3-benzodioxol-5-yl)-N-[6-(3-acetamidophenyl)-5-methylpyridin-2-yl]-2-methylpropanamide;
3-{6-[2-(2,2-difluoro-2H-1,3-benzodioxol-5-yl)-2-methylpropanamido]-3-methylpyridin-2-yl}-4-methoxybenzoic acid;
3-{6-[2-(2,2-difluoro-2H-1,3-benzodioxol-5-yl)-2-methylpropanamido]-3-methylpyridin-2-yl}-5-methoxybenzoic acid;
3-{6-[2-(2,2-difluoro-2H-1,3-benzodioxol-5-yl)-2-methylpropanamido]-3-methylpyridin-2-yl}-2-methoxybenzoic acid;
5-{6-[2-(2,2-difluoro-2H-1,3-benzodioxol-5-yl)-2-methylpropanamido]-3-methylpyridin-2-yl}-2-methoxybenzoic acid;
methyl N-(3-{6-[2-(2,2-difluoro-2H-1,3-benzodioxol-5-yl)-2-methylpropanamido]-3-methylpyridin-2-yl}phenyl)carbamate;
2-(2,2-difluoro-2H-1,3-benzodioxol-5-yl)-N-[6-(3-methanesulfonamidophenyl)-5-methylpyridin-2-yl]-2-methylpropanamide;
2-(2,2-difluoro-2H-1,3-benzodioxol-5-yl)-2-methyl-N-[5-methyl-6-(3-propanamidophenyl) yridine-2-yl]propanamide;
2-(2,2-difluoro-2H-1,3-benzodioxol-5-yl)-2-methyl-N-{5-methyl-6-[3-(2-methylpropanamido)phenyl] yridine-2-yl}propanamide;
ethyl N-(3-{6-[2-(2,2-difluoro-2H-1,3-benzodioxol-5-yl)-2-methylpropanamido]-3-methylpyridin-2-yl}phenyl)carbamate;
2-(2,2-difluoro-2H-1,3-benzodioxol-5-yl)-N-[6-(3-acetamido-4-fluorophenyl)-5-methylpyridin-2-yl]-2-methylpropanamide;
propan-2-yl N-(3-{6-[2-(2,2-difluoro-2H-1,3-benzodioxol-5-yl)-2-methylpropanamido]-3-methylpyridin-2-yl}phenyl)carbamate;
2-(2,2-difluoro-2H-1,3-benzodioxol-5-yl)-N-[6-(3-ethanesulfonamidophenyl)-5-methylpyridin-2-yl]-2-methylpropanamide;
2-(2,2-difluoro-2H-1,3-benzodioxol-5-yl)-N-(6-{3-[(dimethylcarbamoyl)amino]phenyl}-5-methylpyridin-2-yl)-2-methylpropanamide; and
5-{6-[2-(2,2-difluoro-2H-1,3-benzodioxol-5-yl)-2-methylpropanamido]-3-methylpyridin-2-yl}-2-methylbenzoic acid.

In one embodiment, the secondary active agent of the composition is selected from the following
N-[6-(1H-1,2,3-benzotriazol-5-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-2-methyl-N-[5-methyl-6-(2-oxo-2,3-dihydro-1H-indol-5-yl)pyridin-2-yl]propanamide;
2-(2,2-difluoro-2H-1,3-benzodioxol-5-yl)-2-methyl-N-[5-methyl-6-(2-oxo-2,3-dihydro-1H-indol-6-yl)pyridin-2-yl]propanamide;
N-[6-(1,3-benzoxazol-5-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-N-[6-(1H-indazol-6-yl)-5-methylpyridin-2-yl]-2-methylpropanamide;
2-(2,2-difluoro-2H-1,3-benzodioxol-5-yl)-N-[6-(1H-indazol-5-yl)-5-methylpyridin-2-yl]-2-methylpropanamide;
2-(2,2-difluoro-2H-1,3-benzodioxol-5-yl)-2-methyl-N-[5-methyl-6-(3-methyl-1H-indazol-5-yl)pyridin-2-yl]propanamide;
2-(2,2-difluoro-2H-1,3-benzodioxol-5-yl)-2-methyl-N-[5-methyl-6-(1-methyl-1H-indazol-5-yl)pyridin-2-yl]propanamide;
2-(2,2-difluoro-2H-1,3-benzodioxol-5-yl)-2-methyl-N-[5-methyl-6-(1-methyl-1H-indazol-6-yl)pyridin-2-yl]propanamide;
2-(2,2-difluoro-2H-1,3-benzodioxol-5-yl)-N-[6-(1H-indol-6-yl)-5-methylpyridin-2-yl]-2-methylpropanamide;
2-(2,2-difluoro-2H-1,3-benzodioxol-5-yl)-2-methyl-N-[5-methyl-6-(2-oxo-2,3-dihydro-1,3-benzoxazol-5-yl)pyridin-2-yl]propanamide;
2-(2,2-difluoro-2H-1,3-benzodioxol-5-yl)-2-methyl-N-[5-methyl-6-(2-oxo-2,3-dihydro-1H-1,3-benzodiazol-5-yl)pyridin-2-yl]propanamide;
N-[6-(1,2-benzoxazol-6-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-2-methyl-N-[5-methyl-6-(3-methyl-1H-indazol-6-yl)pyridin-2-yl]propanamide;
N-[6-(2H-1,3-benzodioxol-5-yl)-5-methylpyridin-2-yl]-2-(2,2-difluoro-2H-1,3-benzodioxol-5-yl)-2-methylpropanamide;
N-[6-(3,3-difluoro-2-oxo-2,3-dihydro-1H-indol-6-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-2-methyl-N-[5-methyl-6-(3-oxo-2,3-dihydro-1H-isoindol-5-yl)pyridin-2-yl]propanamide;
N-[6-(1,3-benzothiazol-5-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-2-methyl-N-[5-methyl-6-(2-oxo-2,3-dihydro-1,3-benzoxazol-6-yl)pyridin-2-yl]propanamide;
2-(2,2-difluoro-2H-1,3-benzodioxol-5-yl)-N-[6-(3,3-dimethyl-2-oxo-2,3-dihydro-1H-indol-6-yl)-5-methylpyridin-2-yl]-2-methylpropanamide;
2-(2,2-difluoro-2H-1,3-benzodioxol-5-yl)-2-methyl-N-[5-methyl-6-(2-oxo-1,2,3,4-tetrahydroquinolin-7-yl)pyridin-2-yl]propanamide;
2-(2,2-difluoro-2H-1,3-benzodioxol-5-yl)-2-methyl-N-[5-methyl-6-(2-oxo-2,4-dihydro-1H-3,1-benzoxazin-7-yl)pyridin-2-yl]propanamide;
2-(2,2-difluoro-2H-1,3-benzodioxol-5-yl)-2-methyl-N-[5-methyl-6-(3-oxo-3,4-dihydro-2H-1,4-benzoxazin-6-yl)pyridin-2-yl]propanamide;
2-(2,2-difluoro-2H-1,3-benzodioxol-5-yl)-2-methyl-N-[5-methyl-6-(2-methyl-2H-indazol-6-yl)pyridin-2-yl]propanamide;
2-(2,2-difluoro-2H-1,3-benzodioxol-5-yl)-2-methyl-N-[5-methyl-6-(1-methyl-1H-1,3-benzodiazol-5-yl)pyridin-2-yl]propanamide;
2-(2,2-difluoro-2H-1,3-benzodioxol-5-yl)-2-methyl-N-[5-methyl-6-(1-oxo-1,2-dihydroisoquinolin-7-yl)pyridin-2-yl]propanamide;
2-(2,2-difluoro-2H-1,3-benzodioxol-5-yl)-N-[6-(2,3-dihydro-1-benzofuran-6-yl)-5-methylpyridin-2-yl]-2-methylpropanamide;
2-(2,2-difluoro-2H-1,3-benzodioxol-5-yl)-2-methyl-N-[5-methyl-6-(4-oxo-1,4-dihydroquinazolin-7-yl)pyridin-2-yl]propanamide
2-(2,2-difluoro-2H-1,3-benzodioxol-5-yl)-2-methyl-N-[5-methyl-6-(1-methyl-2-oxo-2,3-dihydro-1H-indol-6-yl)pyridin-2-yl]propanamide;
2-(2,2-difluoro-2H-1,3-benzodioxol-5-yl)-2-methyl-N-[5-methyl-6-(4-methyl-3,4-dihydro-2H-1,4-benzoxazin-7-yl)pyridin-2-yl]propanamide;
2-(2,2-difluoro-2H-1,3-benzodioxol-5-yl)-2-methyl-N-[5-methyl-6-(3-methyl-2-oxo-2,3-dihydro-1,3-benzoxazol-6-yl)pyridin-2-yl]propanamide;
2-(2,2-difluoro-2H-1,3-benzodioxol-5-yl)-N-{6-[3-(hydroxymethyl)-1H-indazol-6-yl]-5-methylpyridin-2-yl}-2-methylpropanamide;
2-(2,2-difluoro-2H-1,3-benzodioxol-5-yl)-N-{6-[1-(2-hydroxyethyl)-1H-indazol-6-yl]-5-methylpyridin-2-yl}-2-methylpropanamide;
2-(2,2-difluoro-2H-1,3-benzodioxol-5-yl)-N-{6-[3-(hydroxymethyl)-1H-indazol-5-yl]-5-methylpyridin-2-yl}-2-methylpropanamide;
6-{6-[2-(2,2-difluoro-2H-1,3-benzodioxol-5-yl)-2-methylpropanamido]-3-methylpyridin-2-yl}-1H-indazole-3-carboxylic acid;
2-(2,2-difluoro-2H-1,3-benzodioxol-5-yl)-2-methyl-N-[5-methyl-6-(3-methyl-2-oxo-2,3-dihydro-1,3-benzoxazol-5-yl)pyridin-2-yl]propanamide;
2-(2,2-difluoro-2H-1,3-benzodioxol-5-yl)-N-[6-(3-hydroxy-3-methyl-2-oxo-2,3-dihydro-1H-indol-6-yl)-5-methylpyridin-2-yl]-2-methylpropanamide;
2-(5-{6-[2-(2,2-difluoro-2H-1,3-benzodioxol-5-yl)-2-methylpropanamido]-3-methylpyridin-2-yl}-1H-indazol-1-yl)acetic acid;
2-(2,2-difluoro-2H-1,3-benzodioxol-5-yl)-2-methyl-N-[5-methyl-6-(1-methyl-2-oxo-2,3-dihydro-1H-1,3-benzodiazol-5-yl)pyridin-2-yl]propanamide;
2-(2,2-difluoro-2H-1,3-benzodioxol-5-yl)-N-{6-[1-(2-hydroxyethyl)-1H-indazol-5-yl]-5-methylpyridin-2-yl}-2-methylpropanamide;
2-(2,2-difluoro-2H-1,3-benzodioxol-5-yl)-2-methyl-N-[5-methyl-6-(3-methyl-2-oxo-2,3-dihydro-1H-1,3-benzodiazol-5-yl)pyridin-2-yl]propanamide;
2-(2H-1,3-benzodioxol-5-yl)-2-methyl-N-[5-methyl-6-(3-methyl-1H-indazol-5-yl)pyridin-2-yl]propanamide;
2-(2H-1,3-benzodioxol-5-yl)-N-[6-(1H-indol-5-yl)-5-methylpyridin-2-yl]-2-methylpropanamide;
2-(2H-1,3-benzodioxol-5-yl)-2-methyl-N-[5-methyl-6-(3-methyl-1H-indazol-6-yl)pyridin-2-yl]propanamide;
2-(2H-1,3-benzodioxol-5-yl)-N-[6-(1H-indol-6-yl)-5-methylpyridin-2-yl]-2-methylpropanamide;
2-(2,2-difluoro-2H-1,3-benzodioxol-5-yl)-N-[6-(2,2-dimethyl-2,3-dihydro-1H-indol-6-yl)-5-methylpyridin-2-yl]-2-methylpropanamide;
N-[6-(2,1-benzothiazol-6-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-N-[6-(1,3-dimethyl-1H-indazol-5-yl)-5-methylpyridin-2-yl]-2-methylpropanamide.
2-(2,2-difluoro-2H-1,3-benzodioxol-5-yl)-2-methyl-N-(5-methyl-6-{1-methyl-1H-pyrazolo[3,4-c]pyridin-5-yl}pyridin-2-yl)propanamide;
2-(2,2-difluoro-2H-1,3-benzodioxol-5-yl)-2-methyl-N-(5-methyl-6-{1H-pyrazolo[4,3-b]pyridin-6-yl}pyridin-2-yl)propanamide;
N-[6-(6-chloro-2-oxo-2,3-dihydro-1H-indol-5-yl)-5-methylpyridin-2-yl]-2-(2,2-difluoro-2H-1,3-benzodioxol-5-yl)-2-methylpropanamide;
2-(2,2-difluoro-2H-1,3-benzodioxol-5-yl)-N-[6-(7-fluoro-2-oxo-2,3-dihydro-1H-indol-5-yl)-5-methylpyridin-2-yl]-2-methylpropanamide; and
N-[6-(7-chloro-2-oxo-2,3-dihydro-1H-indol-5-yl)-5-methylpyridin-2-yl]-2-(2,2-difluoro-2H-1,3-benzodioxol-5-yl)-2-methylpropanamide.

### B. Definitions

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the term “bioactivity” indicates an effect on one or more cellular or extracellular process (e.g., via binding, signaling, etc.) which can impact physiological or pathophysiological processes.

As used herein, N-oxide, or amine oxide, refers to a compound derived from a tertiary amine by the attachment of one oxygen atom to the nitrogen atom, R₃N⁺—O⁻. By extension the term includes the analogous derivatives of primary and secondary amines.

As used herein, “protein” is used synonymously with “peptide,” “polypeptide,” or “peptide fragment”. A “purified” polypeptide, protein, peptide, or peptide fragment is substantially free of cellular material or other contaminating proteins from the cell, tissue, or cell-free source from which the amino acid sequence is obtained, or substantially free from chemical precursors or other chemicals when chemically synthesized.

As used here, the terms “nitric oxide” and “NO” encompass uncharged nitric oxide and charged nitric oxide species, particularly including nitrosonium ion (NO⁺) and nitroxyl ion (NO⁻). The reactive form of nitric oxide can be provided by gaseous nitric oxide. Compounds having the structure X—NO_{y} wherein X is a nitric oxide releasing, delivering, or transferring moiety, including any and all such compounds which provide nitric oxide to its intended site of action in a form active for their intended purpose, and Y is 1 or 2.

“Repair” means recovering of structural integrity and normal physiologic function. By way of example, the oral and upper airway respiratory epithelium can repair damage done by thermal injury or viral infection.

“Regeneration” means the ability of an organ to enter non-malignant cellular, vascular and stromal growth to restore functional organ tissue. By way of example, wound healing involves regeneration of tissue and organs (e.g. skin, gastric and intestinal mucosa), as does bone following fracture, and the liver following partial surgical removal and exposure to infectious or toxic insult.

As utilized herein, the term “pharmaceutically acceptable” means approved by a regulatory agency of a federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils.

A “pharmaceutically acceptable salt” or “salt” of a compound of the invention is a product of the disclosed compound that contains an ionic bond, and is typically produced by reacting the disclosed compound with either an acid or a base, suitable for administering to a subject. A pharmaceutically acceptable salt can include, but is not limited to, acid addition salts including hydrochlorides, hydrobromides, phosphates, sulphates, hydrogen sulphates, alkylsulphonates, arylsulphonates, arylalkylsulfonates, acetates, benzoates, citrates, maleates, fumarates, succinates, lactates, and tartrates; alkali metal cations such as Li, Na, and K, alkali earth metal salts such as Mg or Ca, or organic amine salts.

A “pharmaceutical composition” is a formulation comprising the disclosed combination in a form suitable for administration to a subject. A pharmaceutical composition of the invention is preferably formulated to be compatible with its intended route of administration. Examples of routes of administration include, but are not limited to, oral and parenteral, e.g., intravenous, intradermal, subcutaneous, inhalation, topical, transdermal, transmucosal, and rectal administration.

As used herein, a “secondary active agent” is a compound or therapy that increases CFTR function. In one embodiment, a secondary active agent is selected from the group consisting of CFTR correctors and CFTR potentiators. In one embodiment, a secondary active agent is selected from the group consisting of CFTR correctors, potentiators, or amplifiers as well as gene therapy directed toward CF.

As used herein, a “CFTR corrector” is a compound that promotes maturation and delivery of CFTR proteins to the apical surface. Examples of CFTR correctors include but are not limited to VX-809 (3-{6-{[1-(2,2-difluoro-1,3-benzodioxol-5-yl)cyclopropanecarbonyl]amino}-3-methylpyridin-2-yl}benzoic acid), VX-661 (1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide), compounds of PCT/US2014/038385 and compounds of PCT/US2015/021841.

VX-809 has also recently been classified as a “CFTR conformational stabilizer” by the FDA in a Summary Review of Regulatory Action dated Jun. 25, 2015, but continues to be know in the art and literature as a CFTR corrector, and is treated as such herein.

As used herein, a “CFTR potentiator” is a compound that activates apical CFTR by increasing the open time of the channel. An example of a CFTR potentiator includes but is not limited to VX-770 (N-(2,4-Di-tert-butyl-5-hydroxyphenyl)-4-oxo-1,4-dihydroquinoline-3-carboxamide).

As used herein, “gene therapy” is any therapy directed toward the genetic defect in CF.

As used herein a “CFTR amplifier” is any compound that increases CFTR activity.

A “palliative care agent” is an agent for the management of CF other than a secondary active agent that may include a mucolytic agent, a bronchodilator, an antibiotic, an anti-infective agent, an anti-inflammatory agent, a nutritional agent, or other agent known to manage the symptoms of CF, collectively termed herein as palliative care.

“Stable compound” and “stable structure” are meant to indicate a compound that is sufficiently robust to survive isolation to a useful degree of purity from a reaction mixture, and formulation into an efficacious therapeutic agent.

As used herein the term “therapeutically effective amount” generally means the amount necessary to ameliorate at least one symptom of a disorder to be prevented, reduced, or treated as described herein. The phrase “therapeutically effective amount” as it relates to the GSNOR inhibitors of the present invention shall mean the GSNOR inhibitor dosage that provides the specific pharmacological response for which the GSNOR inhibitor is administered in a significant number of subjects in need of such treatment. It is emphasized that a therapeutically effective amount of a GSNOR inhibitor that is administered to a particular subject in a particular instance will not always be effective in treating the conditions/diseases described herein, even though such dosage is deemed to be a therapeutically effective amount by those of skill in the art.

The phrase “therapeutically effective amount” as it relates to the secondary active agent of the present invention shall mean the dosage that provides the specific pharmacological response for which the secondary active agent is administered in a significant number of subjects in need of such treatment.

The term “biological sample” includes, but is not limited to, samples of blood (e.g., serum, plasma, or whole blood), urine, saliva, sweat, breast milk, vaginal secretions, semen, hair follicles, skin, teeth, bones, nails, or other secretions, body fluids, tissues, or cells. In accordance with the invention, the levels of the GSNO in the biological sample can be determined by the methods described in U.S. Patent Application Publication No. 2005/0014697.

### C. Pharmaceutical Compositions

Pharmaceutical compositions of the invention include compositions comprising a GSNOR inhibitor in combination with one or more secondary active agents.

In one embodiment, the pharmaceutical composition of the invention comprises:
i) a GSNOR inhibitor or a pharmaceutically acceptable salt thereof, in combination with
ii) one or more secondary active agents selected from the group consisting of CFTR correctors and CFTR potentiators or pharmaceutically acceptable salt(s) thereof.

The GSNOR inhibitor of the pharmaceutical composition can be administered concurrently with, prior to, or subsequent to, one or more secondary active agents.

In another embodiment, the pharmaceutical composition can be administered concurrently with, prior to, or subsequent to, one or more palliative care agents.

In another embodiment, the GSNOR inhibitor of the invention can be administered concurrently with, prior to, or subsequent to, one or more secondary active agents or palliative care agents.

The invention encompasses pharmaceutical compositions comprising the compositions described herein and at least one pharmaceutically acceptable carrier. Suitable carriers are described in “Remington: The Science and Practice, Twentieth Edition,” published by Lippincott Williams & Wilkins, which is incorporated herein by reference. Pharmaceutical compositions according to the invention may also comprise one or more non-inventive compound active agents.

The pharmaceutical compositions of the invention can comprise compounds described herein, the pharmaceutical compositions can comprise known compounds which previously were not known to have GSNOR inhibitor activity, or a combination thereof.

The compounds of the pharmacecutical compositions of the present invention can be employed in combination therapies, that is, the compounds and pharmaceutically acceptable compositions can be administered concurrently with, prior to, or subsequent to, one or more other desired secondary active agents or medical procedures. The particular combination of therapies (secondary agents or procedures) to employ in a combination regimen will take into account compatibility of the desired agents and/or procedures and the desired therapeutic effect to be achieved. The therapies employed may achieve a desired effect for the same disorder (for example, a pharmaceutical composition may be administered concurrently with one or more secondary agents used to treat the same disorder), or they may achieve different effects (such as control adverse effects).

In one embodiment the secondary active agent of the pharmaceutical combination is selected from a compound or therapy that increases CFTR function. In one embodiment, the secondary active agent is selected from a CFTR corrector or a CFTR potentiator.

In one embodiment, the pharmaceutical composition may be used with any single or combination of palliative agents including mucolytic agents, bronchodilators, antibiotics, anti-infective agents, an anti-inflammatory agents, a nutritional agents, or other palliative agents known to manage CF.

The compounds of the pharmaceutical combination of the invention can be utilized in any pharmaceutically acceptable dosage form, including, but not limited to injectable dosage forms, liquid dispersions, gels, aerosols, ointments, creams, lyophilized formulations, dry powders, tablets, capsules, controlled release formulations, fast melt formulations, delayed release formulations, extended release formulations, pulsatile release formulations, mixed immediate release and controlled release formulations, etc. Specifically, the compounds of the invention described herein can be formulated: (a) for administration selected from the group consisting of oral, pulmonary, intravenous, intra-arterial, intrathecal, intra-articular, rectal, ophthalmic, colonic, parenteral, intracisternal, intravaginal, intraperitoneal, local, buccal, nasal, and topical administration; (b) into a dosage form selected from the group consisting of liquid dispersions, gels, aerosols, ointments, creams, tablets, sachets, and capsules; (c) into a dosage form selected from the group consisting of lyophilized formulations, dry powders, fast melt formulations, controlled release formulations, delayed release formulations, extended release formulations, pulsatile release formulations, and mixed immediate release and controlled release formulations; or (d) any combination thereof.

For respiratory infections or pulmonary exacerbations of CF, an inhalation formulation can be used to achieve high local concentrations. Formulations suitable for inhalation include dry power or aerosolized or vaporized solutions, dispersions, or suspensions capable of being dispensed by an inhaler or nebulizer into the endobronchial or nasal cavity of infected patients to treat upper and lower respiratory bacterial infections.

Solutions or suspensions used for parenteral, intradermal, or subcutaneous application can comprise one or more of the following components: (1) a sterile diluent such as water for injection, saline solution, fixed oils, polyethylene glycols, glycerine, propylene glycol, or other synthetic solvents; (2) antibacterial agents such as benzyl alcohol or methyl parabens; (3) antioxidants such as ascorbic acid or sodium bisulfite; (4) chelating agents such as ethylenediaminetetraacetic acid; (5) buffers such as acetates, citrates, or phosphates; and (5) agents for the adjustment of tonicity such as sodium chloride or dextrose. The pH can be adjusted with acids or bases, such as hydrochloric acid or sodium hydroxide. A parenteral preparation can be enclosed in ampoules, disposable syringes, or multiple dose vials made of glass or plastic.

Pharmaceutical compositions suitable for injectable use may comprise sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.), or phosphate buffered saline (PBS). In all cases, the composition must be sterile and should be fluid to the extent that easy syringability exists. The pharmaceutical composition should be stable under the conditions of manufacture and storage and should be preserved against the contaminating action of microorganisms such as bacteria and fungi.

The carrier can be a solvent or dispersion medium comprising, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol or sorbitol, and inorganic salts such as sodium chloride in the composition. Prolonged absorption of the injectable compositions can be brought about by including in the composition an agent which delays absorption, for example, aluminum monostearate and gelatin.

Sterile injectable solutions can be prepared by incorporating the active reagent in the required amount in an appropriate solvent with one or a combination of ingredients enumerated above, as required, followed by filtered sterilization. Generally, dispersions are prepared by incorporating at least one compound of the invention into a sterile vehicle that contains a basic dispersion medium and any other required ingredients. In the case of sterile powders for the preparation of sterile injectable solutions, exemplary methods of preparation include vacuum drying and freeze-drying, both of which yield a powder of a compound of the invention plus any additional desired ingredient from a previously sterile-filtered solution thereof.

Oral compositions generally include an inert diluent or an edible carrier. They can be enclosed, for example, in gelatin capsules or compressed into tablets. For the purpose of oral therapeutic administration, the compound of the invention can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition.

For administration by inhalation, the compounds are delivered in the form of an aerosol spray from pressured container or dispenser that contains a suitable propellant, e.g., a gas such as carbon dioxide, a nebulized liquid, or a dry powder from a suitable device. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active reagents are formulated into ointments, salves, gels, or creams as generally known in the art. The reagents can also be prepared in the form of suppositories (e.g., with conventional suppository bases such as cocoa butter and other glycerides) or retention enemas for rectal delivery.

In one embodiment, the compounds of the invention are prepared with carriers that will protect against rapid elimination from the body. For example, a controlled release formulation can be used, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Additionally, suspensions of the compounds of the invention may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also include suitable stabilizers or agents to increase the solubility of the compounds and allow for the preparation of highly concentrated solutions.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of the compound of the invention calculated to produce the desired therapeutic effect in association with the required pharmaceutical carrier. The specification for the dosage unit forms of the invention are dictated by and directly dependent on the unique characteristics of the compound of the invention and the particular therapeutic effect to be achieved, and the limitations inherent in the art of compounding such an active agent for the treatment of individuals.

Pharmaceutical compositions according to the invention can comprise one or more pharmaceutical excipients. Examples of such excipients include, but are not limited to binding agents, filling agents, lubricating agents, suspending agents, sweeteners, flavoring agents, preservatives, buffers, wetting agents, disintegrants, effervescent agents, and other excipients. Such excipients are known in the art. Exemplary excipients include: (1) binding agents which include various celluloses and cross-linked polyvinylpyrrolidone, microcrystalline cellulose, such as Avicel® PH101 and Avicel® PH102, silicified microcrystalline cellulose (ProSolv SMCC™), gum tragacanth and gelatin; (2) filling agents such as various starches, lactose, lactose monohydrate, and lactose anhydrous; (3) disintegrating agents such as alginic acid, Primogel, corn starch, lightly crosslinked polyvinyl pyrrolidone, potato starch, maize starch, and modified starches, croscarmellose sodium, cross-povidone, sodium starch glycolate, and mixtures thereof; (4) lubricants, including agents that act on the flowability of a powder to be compressed, include magnesium stearate, colloidal silicon dioxide, such as Aerosil® 200, talc, stearic acid, calcium stearate, and silica gel; (5) glidants such as colloidal silicon dioxide; (6) preservatives, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride; (7) diluents such as pharmaceutically acceptable inert fillers, such as microcrystalline cellulose, lactose, dibasic calcium phosphate, saccharides, and/or mixtures of any of the foregoing; examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose; (8) sweetening agents, including any natural or artificial sweetener, such as sucrose, saccharin sucrose, xylitol, sodium saccharin, cyclamate, aspartame, and acesulfame; (9) flavoring agents, such as peppermint, methyl salicylate, orange flavoring, Magnasweet® (trademark of MAFCO), bubble gum flavor, fruit flavors, and the like; and (10) effervescent agents, including effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

### D. Kits Comprising the Compositions of the Invention

The present invention also encompasses kits comprising the compositions of the invention. Such kits can comprise, for example, (1) at least one compound of the invention; and (2) at least one pharmaceutically acceptable carrier, such as a solvent or solution. Additional kit components can optionally include, for example: (1) any of the pharmaceutically acceptable excipients identified herein, such as stabilizers, buffers, etc., (2) at least one container, vial, or similar apparatus for holding and/or mixing the kit components; and (3) delivery apparatus, such as an inhaler, nebulizer, syringe, etc.

### E. Methods of Treatment

The invention encompasses methods of preventing or treating (e.g., alleviating) one or more symptoms of cystic fibrosis through the use of one or more of the disclosed pharmaceutical compositions. The methods comprise administering a therapeutically effective amount of a GSNOR inhibitor in combination with one or more secondary agent(s) to a patient in need. The GSNOR inhibitor can be administered concurrently with, prior to, or subsequent to, one or more secondary active agents.

The compositions of the invention can also be used for prophylactic therapy. In one embodiment, the method is a method of treating or lessening the severity of cystic fibrosis in a patient, comprising the step of administering to said patient an effective amount of the pharmaceutical composition described herein.

The GSNOR inhibitor of the pharmaceutical composition of the invention used in the methods of treatment according to the invention can be: (1) a GSNOR inhibitor compound described herein, or a pharmaceutically acceptable salt thereof, a stereoisomer thereof, a prodrug thereof, a metabolite thereof, or an N-oxide thereof; (2) a compound which was known prior to the present invention, but wherein it was not known that the compound is a GSNOR inhibitor, or a pharmaceutically acceptable salt thereof, a stereoisomer thereof, a prodrug thereof, a metabolite thereof, or an N-oxide thereof; or (3) a compound which was known prior to the present invention, and wherein it was known that the compound is a GSNOR inhibitor, but wherein it was not known that the compound is useful for the methods of treatment described herein, or a pharmaceutically acceptable salt, a stereoisomer, a prodrug, a metabolite, or an N-oxide thereof.

The methods of the present invention can be pharmaceutical compositions of the invention employed in combination therapies, that is, the pharmaceutical compositions can be administered concurrently with, prior to, or subsequent to, one or more other desired secondary active agents or medical procedures. The particular combination of therapies (secondary agents or procedures) to employ in a combination regimen will take into account compatibility of the desired agents and/or procedures and the desired therapeutic effect to be achieved.

The patient can be any animal, domestic, livestock, or wild, including, but not limited to cats, dogs, horses, pigs, and cattle, and preferably human patients. As used herein, the terms patient and subject may be used interchangeably.

As used herein, “treating” describes the management and care of a patient for the purpose of combating a disease, condition, or disorder and includes the administration of a compound of the present invention to prevent the onset of the symptoms or complications, alleviating the symptoms or complications, or eliminating the disease, condition, or disorder. More specifically, “treating” includes reversing, attenuating, alleviating, minimizing, suppressing, or halting at least one deleterious symptom or effect of a disease (disorder) state, disease progression, disease causative agent (e.g., bacteria or viruses), or other abnormal condition. Treatment is continued as long as symptoms and/or pathology ameliorate.

In general, the dosage, i.e., the therapeutically effective amount, ranges from 1 g/kg to 10 g/kg and often ranges from 10 μg/kg to 1 g/kg or 10 μg/kg to 100 mg/kg body weight of the subject being treated, per day.

### F. Uses

In subjects with deleteriously high levels of GSNOR or GSNOR activity, modulation may be achieved, for example, by administering one or more of the GSNOR inhibitors of the disclosed compositions that disrupts or down-regulates GSNOR function, or decreases GSNOR levels. These compounds may be administered with other GSNOR inhibitor agents, such as anti-GSNOR antibodies or antibody fragments, GSNOR antisense, iRNA, or small molecules, or other inhibitors, alone or in combination with other agents as described in detail herein.

The present invention provides a method of treating a subject afflicted with a disorder ameliorated by NO donor therapy. Such a method comprises administering to a subject a therapeutically effective amount of a GSNOR inhibitor in combination with one or more secondary active agents.

The disorders can include pulmonary disorders associated with hypoxemia and/or smooth muscle constriction in the lungs and airways and/or lung infection and/or lung inflammation and/or lung injury (e.g., pulmonary hypertension, ARDS, asthma, pneumonia, pulmonary fibrosis/interstitial lung diseases, cystic fibrosis, COPD); cardiovascular disease and heart disease (e.g., hypertension, ischemic coronary syndromes, atherosclerosis, heart failure, glaucoma); diseases characterized by angiogenesis (e.g., coronary artery disease); disorders where there is risk of thrombosis occurring; disorders where there is risk of restenosis occurring; inflammatory diseases (e.g., AIDS related dementia, inflammatory bowel disease (IBD), Crohn's disease, colitis, and psoriasis); functional bowel disorders (e.g., irritable bowel syndrome (IBS)); diseases where there is risk of apoptosis occurring (e.g., heart failure, atherosclerosis, degenerative neurologic disorders, arthritis, and liver injury (e.g., drug induced, ischemic or alcoholic)); impotence; sleep apnea; diabetic wound healing; cutaneous infections; treatment of psoriasis; obesity caused by eating in response to craving for food; stroke; reperfusion injury (e.g., traumatic muscle injury in heart or lung or crush injury); and disorders where preconditioning of heart or brain for NO protection against subsequent ischemic events is beneficial, central nervous system (CNS) disorders (e.g., anxiety, depression, psychosis, and schizophrenia); and infections caused by bacteria (e.g., tuberculosis, C. difficile infections, among others).

In one embodiment, the disorder is cystic fibrosis. Pharmaceutical compositions of the invention are capable of treating and/or slowing the progression of cystic fibrosis. For approximately 90% of patients with CF, death results from progressive respiratory failure associated with impaired mucus clearance and excessive overgrowth of bacteria and fungi in the airways (Gibson et al., 2003, Proesmans et al., 2008). GSNOR inhibitors are capable of preserving endogenous s-nitrosothiol (SNO) pools via inhibiting GSNO catabolism and therefore may positively modulate CFTR. GSNOR inhibitors are distinguished by their ability to demonstrate preservation of GSNO, potent bronchodilatory and anti-inflammatory effects in animal models of COPD (porcine pancreatic elastase) (Blonder et al., ATS 2011 abstract reference) and asthma. Pharmacuetical compositions of the invention are capable of treating and/or slowing the progression of CF. In this embodiment, appropriate amounts of compounds of the pharmaceutical compositions are an amount sufficient to treat and/or slow the progression of CF and can be determined without undue experimentation by preclinical and/or clinical trials.

In one embodiment, the compounds of the pharmaceutical compositions of the present invention or a pharmaceutically acceptable salt thereof, or a prodrug, stereoisomer, metabolite, or N-oxide thereof, can be administered in combination with an NO donor. An NO donor donates nitric oxide or a related redox species and more generally provides nitric oxide bioactivity, that is activity which is identified with nitric oxide, e.g., vasorelaxation or stimulation or inhibition of a receptor protein, e.g., ras protein, adrenergic receptor, NFκB. NO donors including S-nitroso, O-nitroso, C-nitroso, and N-nitroso compounds and nitro derivatives thereof and metal NO complexes, but not excluding other NO bioactivity generating compounds, useful herein are described in “Methods in Nitric Oxide Research,” Feelisch et al. eds., pages 71-115 (J. S., John Wiley & Sons, New York, 1996), which is incorporated herein by reference. NO donors which are C-nitroso compounds where nitroso is attached to a tertiary carbon which are useful herein include those described in U.S. Pat. No. 6,359,182 and in WO 02/34705. Examples of S-nitroso compounds, including S-nitrosothiols useful herein, include, for example, S-nitrosoglutathione, S-nitroso-N-acetylpenicillamine, S-nitroso-cysteine and ethyl ester thereof, S-nitroso cysteinyl glycine, S-nitroso-gamma-methyl-L-homocysteine, S-nitroso-L-homocysteine, S-nitroso-gamma-thio-L-leucine, S-nitroso-delta-thio-L-leucine, and S-nitrosoalbumin. Examples of other NO donors useful herein are sodium nitroprusside (nipride), ethyl nitrite, isosorbide, nitroglycerin, SIN 1 which is molsidomine, furoxamines, N-hydroxy (N-nitrosamine), and perfluorocarbons that have been saturated with NO or a hydrophobic NO donor.

In one embodiment, the compounds of the pharmaceutical compositions of the present invention or a pharmaceutically acceptable salt thereof, a stereoisomer thereof, a prodrug thereof, a metabolite thereof, or an N-oxide thereof, can be administered in combination with an agent that imposes nitrosative or oxidative stress. Agents for selectively imposing nitrosative stress to inhibit proliferation of pathologically proliferating cells in combination therapy with GSNOR inhibitors herein and dosages and routes of administration therefor include those disclosed in U.S. Pat. No. 6,057,367, which is incorporated herein. Supplemental agents for imposing oxidative stress (i.e., agents that increase GSSG (oxidized glutathione) over GSH (glutathione) ratio or NAD(P) over NAD(P)H ratio or increase thiobarbituric acid derivatives) in combination therapy with GSNOR inhibitors herein include, for example, L-buthionine-S-sulfoximine (BSO), glutathione reductase inhibitors (e.g., BCNU), inhibitors or uncouplers of mitochondrial respiration, and drugs that increase reactive oxygen species (ROS), e.g., adriamycin, in standard dosages with standard routes of administration.

GSNOR inhibitors may also be co-administered with a phosphodiesterase inhibitor (e.g., rolipram, cilomilast, roflumilast, Viagra® (sildenifil citrate), Cialis® (tadalafil), Levitra® (vardenifil), etc.), a β-agonist, a steroid, or a leukotriene antagonist (LTD-4). Those skilled in the art can readily determine the appropriate therapeutically effective amount depending on the disorder to be ameliorated.

GSNOR inhibitors may be used as a means to improve β-adrenergic signaling. In particular, inhibitors of GSNOR alone or in combination with β-agonists could be used to treat or protect against heart failure, or other vascular disorders such as hypertension and asthma. GSNOR inhibitors can also be used to modulate G protein coupled receptors (GPCRs) by potentiating Gs G-protein, leading to smooth muscle relaxation (e.g., airway and blood vessels), and by attenuating Gq G-protein, and thereby preventing smooth muscle contraction (e.g., in airway and blood vessels).

The therapeutically effective amount for the treatment of a subject is the amount that causes amelioration of the disorder being treated or protects against a risk associated with the disorder. For example, for asthma, a therapeutically effective amount is a bronchodilating effective amount; for cystic fibrosis, a therapeutically effective amount is an airway obstruction ameliorating effective amount or an amount effective in lessening the symptoms in the pancreas, GI tract, and/or liver caused by CF; for ARDS, a therapeutically effective amount is a hypoxemia ameliorating effective amount; for heart disease, a therapeutically effective amount is an angina relieving or angiogenesis inducing effective amount; for hypertension, a therapeutically effective amount is a blood pressure reducing effective amount; for ischemic coronary disorders, a therapeutic amount is a blood flow increasing effective amount; for atherosclerosis, a therapeutically effective amount is an endothelial dysfunction reversing effective amount; for glaucoma, a therapeutic amount is an intraocular pressure reducing effective amount; for diseases characterized by angiogenesis, a therapeutically effective amount is an angiogenesis inhibiting effective amount; for disorders where there is risk of thrombosis occurring, a therapeutically effective amount is a thrombosis preventing effective amount; for disorders where there is risk of restenosis occurring, a therapeutically effective amount is a restenosis inhibiting effective amount; for chronic inflammatory diseases, a therapeutically effective amount is an inflammation reducing effective amount; for disorders where there is risk of apoptosis occurring, a therapeutically effective amount is an apoptosis preventing effective amount; for impotence, a therapeutically effective amount is an erection attaining or sustaining effective amount; for obesity, a therapeutically effective amount is a satiety causing effective amount; for stroke, a therapeutically effective amount is a blood flow increasing or a TIA protecting effective amount; for reperfusion injury, a therapeutically effective amount is a function increasing effective amount; and for preconditioning of heart and brain, a therapeutically effective amount is a cell protective effective amount, e.g., as measured by troponin or CPK.

### G. Uses in an Apparatus

The compositions of the present invention or a pharmaceutically acceptable salt thereof, or a stereoisomer, prodrug, metabolite, or N-oxide thereof, can be applied to various apparatus in circumstances when the presence of such compounds would be beneficial. Such apparatus can be any device or container, for example, implantable devices in which a compound of the invention can be used to coat a surgical mesh or cardiovascular stent prior to implantation in a patient. The compounds of the invention can also be applied to various apparatus for in vitro assay purposes or for culturing cells.

The compounds of the compositions of the present invention or a pharmaceutically acceptable salt thereof, or a stereoisomer, a prodrug, a metabolite, or an N-oxide thereof, can also be used as an agent for the development, isolation or purification of binding partners to compounds of the invention, such as antibodies, natural ligands, and the like. Those skilled in the art can readily determine related uses for the compounds of the present invention.

### Examples

The following examples are given to illustrate the present invention. It should be understood, however, that the invention is not to be limited to the specific conditions or details described in these examples. Throughout the specification, any and all references to a publicly available document, including a U.S. patent, are specifically incorporated by reference.

### Example 1: Using Chambers Study of GSNOR Inhibitors (GSNORi) in Combination with CFTR Modulators

Experimental Approach:

In vitro studies were performed using primary HBE cells derived from F508del/F508del CF patients that had undergone lung transplants. Under the guidance of the Cystic Fibrosis Foundation (CFF), lungs were sent to the University of North Carolina, Chapel Hill, Cystic Fibrosis Tissue Procurement Center where HBE cells were isolated following approved and well described protocols (Randell et al 2011). CF HBE cells from two different patients, CFFT010H and CFFT008G were expanded as “Conditionally Reprogrammed Cells” (CRC's, passage 3-4) following published protocols (Liu et al., 2012; Bove, et al., 2014). Expanded CF HBE cells were plated onto porous membrane supports (Snapwells) under air-liquid interface conditions until they formed well-differentiated cultures (presence of ciliated cells, 21-35 days post seeding). To assess modulation in CFTR-mediated Cl⁻ current, Using chamber analyses were conducted under short-circuit conditions (I_{sc}) in bilateral Krebs bicarbonate solution, maintained at 37° C., and continuously aerated with 95% O₂; 5% CO₂. The voltage (mV) was kept clamped at zero. All recordings and calculations were performed by LabChart software.

Experimental Design:

Well-differentiated CF (F508del/F508del) HBE cultures were treated as outlined below:
Vehicle control (DMSO, 0.1%, 24 or 48 h)
CFTR corrector, 3-{6-{[1-(2,2-difluoro-1,3-benzodioxol-5-yl)cyclopropanecarbonyl]amino}-3-methylpyridin-2-yl}benzoic acid (3 μM, 24 h)
CFTR corrector, 3-{6-{[1-(2,2-difluoro-1,3-benzodioxol-5-yl)cyclopropanecarbonyl]amino}-3-methylpyridin-2-yl}benzoic acid (3 μM, 24 h)+GSNORi, 3-chloro-4-(6-hydroxyquinolin-2-yl)benzoic acid (10, 30, 100 μM, 2, 4, 8, 24 h)
CFTR corrector, 3-{6-{[1-(2,2-difluoro-1,3-benzodioxol-5-yl)cyclopropanecarbonyl]amino}-3-methylpyridin-2-yl}benzoic acid (3 μM, 24 or 48 h)+CFTR potentiator, N-(2,4-Di-tert-butyl-5-hydroxyphenyl)-4-oxo-1,4-dihydroquinoline-3-carboxamide (3 M, 24 or 48 h)
CFTR corrector, 3-{6-{[1-(2,2-difluoro-1,3-benzodioxol-5-yl)cyclopropanecarbonyl]amino}-3-methylpyridin-2-yl}benzoic acid (3 μM, 24 or 48 h)+CFTR potentiator, N-(2,4-Di-tert-butyl-5-hydroxyphenyl)-4-oxo-1,4-dihydroquinoline-3-carboxamide (3 M, 24 or 48 h)+GSNORi, 3-chloro-4-(6-hydroxyquinolin-2-yl)benzoic acid (10, 30, 100 μM, 2, 4, 8, 24 h)

Well differentiated CF (F508del/F508del) HBE cultures were mounted into Using chambers (Physiologics Instruments) and the following treatments were performed as outlined below:

Epithelial Na⁺ channel (ENaC) inhibitor (amiloride, 100 μM, apical)
CFTR activator (forskolin, 10 μM, bilateral)
CFTR potentiator (3 μM, apical)
CFTR activator (genistein, 10 μM, apical)
CFTR inhibitor (CFTRinh172, 10 μM, apical)
Ca⁺-activated Cl⁻ channel (CaCC) activator (UTP, 100 μM, apical)

CFTR Modulation with S-Nitrosoglutathione Reductase Inhibitor (GSNORi) 3-chloro-4-(6-hydroxyquinolin-2-yl)benzoic acid in Combination with a CFTR Corrector and with a CFTR Corrector/CFTR Potentiator

Results:

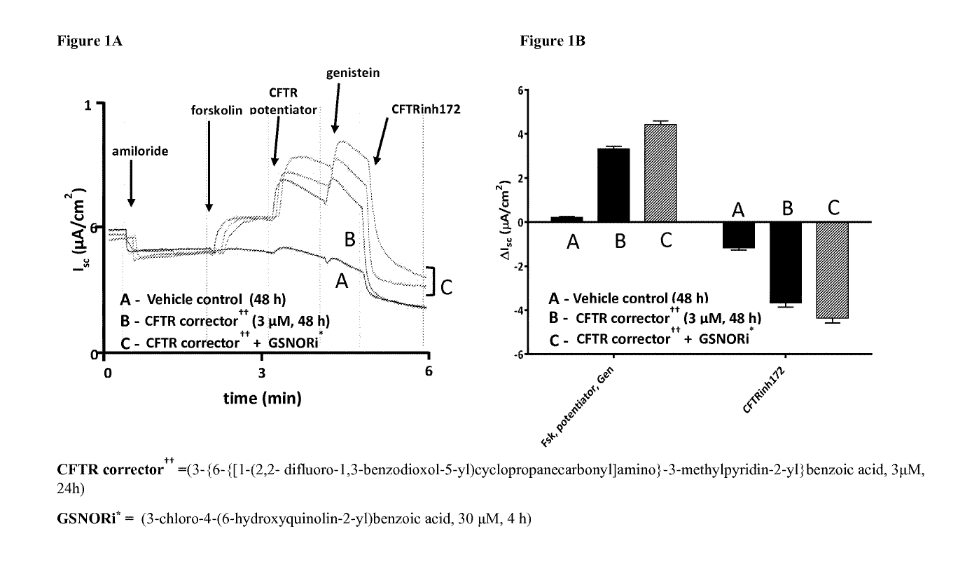

Treatment groups consisted of cells treated with vehicle control (DMSO), cells treated with CFTR corrector (3-{6-{[1-(2,2-difluoro-1,3-benzodioxol-5-yl)cyclopropanecarbonyl]amino}-3-methylpyridin-2-yl}benzoic acid) alone, or cells treated with the combination of CFTR corrector (3-{6-{[1-(2,2-difluoro-1,3-benzodioxol-5-yl)cyclopropanecarbonyl]amino}-3-methylpyridin-2-yl}benzoic acid) and GSNORi (3-chloro-4-(6-hydroxyquinolin-2-yl)benzoic acid). By using chamber analysis, there were no significant changes in overall resistance (Rt, Ω*cm²) and basal short circuit current (I_{sc}) measurements between all groups. The I_{sc} measurements were recorded and analyzed for all three conditions. For the majority of experiments (n=6 runs), a consistent and reproducible increase in CFTR-mediated I_{sc} was observed when 3-chloro-4-(6-hydroxyquinolin-2-yl)benzoic acid was added in combination with 3-{6-{[1-(2,2-difluoro-1,3-benzodioxol-5-yl)cyclopropanecarbonyl]amino}-3-methylpyridin-2-yl}benzoic acid over treatment of 3-{6-{[1-(2,2-difluoro-1,3-benzodioxol-5-yl)cyclopropanecarbonyl]amino}-3-methylpyridin-2-yl}benzoic acid alone. This increase in I_{sc} was confirmed by the observed increase in CFTR-mediated inhibition when CF HBE cells were treated with CFTRᵢₙₕ172, suggesting an overall increase in CFTR activity (FIG. 1A). Quantitation using two separate codes and multiple runs (n=4) showed consistent effects of CFTR activation and inhibition within all three groups (FIG. 1B).

A CFTR agonist-mediated “downward slope” was consistently observed when CF HBE cells were treated with CFTR corrector (3-{6-{[1-(2,2-difluoro-1,3-benzodioxol-5-yl)cyclopropanecarbonyl]amino}-3-methylpyridin-2-yl}benzoic acid) alone. When cells were treated with CFTR corrector 3-{6-{[1-(2,2-difluoro-1,3-benzodioxol-5-yl)cyclopropanecarbonyl]amino}-3-methylpyridin-2-yl}benzoic acid plus GSNORi 3-chloro-4-(6-hydroxyquinolin-2-yl)benzoic acid (optimal response at 30 μM for 4 h), the forskolin-mediated downward slope was less, perhaps through stabilizing CFTR at the cell surface with GSNOR inhibitor. In addition, when the CFTR stimulated area under the curve (AUC) region was measured for all three conditions, an increase in 3-{6-{[1-(2,2-difluoro-1,3-benzodioxol-5-yl)cyclopropanecarbonyl]amino}-3-methylpyridin-2-yl}benzoic acid and 3-{6-{[1-(2,2-difluoro-1,3-benzodioxol-5-yl)cyclopropanecarbonyl]amino}-3-methylpyridin-2-yl}benzoic acid/3-chloro-4-(6-hydroxyquinolin-2-yl)benzoic acid AUC over DMSO negative control was observed. An increase (30-40%) in AUC when CF HBE cells were treated with the 3-{6-{[1-(2,2-difluoro-1,3-benzodioxol-5-yl)cyclopropanecarbonyl]amino}-3-methylpyridin-2-yl}benzoic acid/3-chloro-4-(6-hydroxyquinolin-2-yl)benzoic acid combination over 3-{6-{[1-(2,2-difluoro-1,3-benzodioxol-5-yl)cyclopropanecarbonyl]amino}-3-methylpyridin-2-yl}benzoic acid alone was also observed, suggesting added CFTR-mediated current.

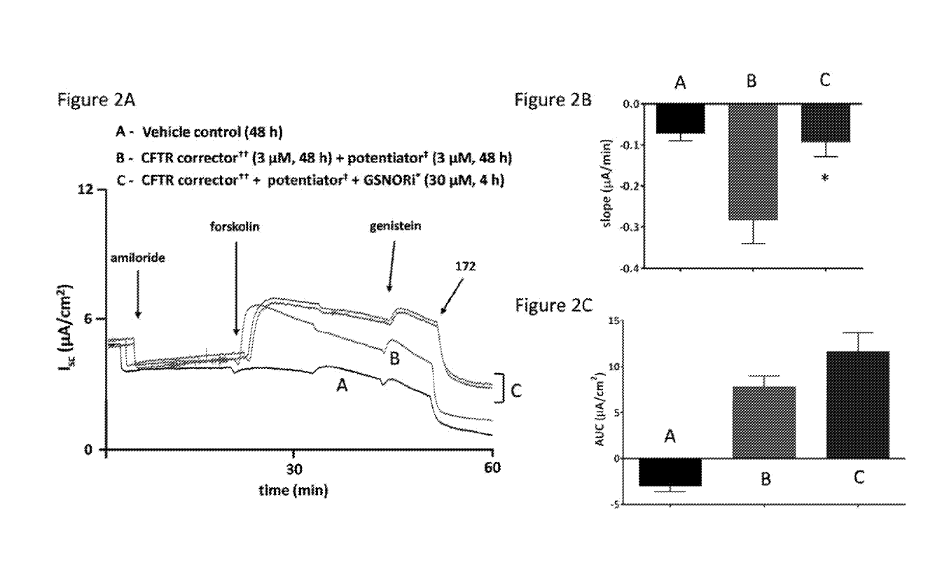

Next, the modulation of CFTR function using CF (F508del/F508del) HBE cells when treated with the triple combination of CFTR corrector (3-{6-{[1-(2,2-difluoro-1,3-benzodioxol-5-yl)cyclopropanecarbonyl]amino}-3-methylpyridin-2-yl}benzoic acid), CFTR potentiator (N-(2,4-Di-tert-butyl-5-hydroxyphenyl)-4-oxo-1,4-dihydroquinoline-3-carboxamide), and GSNORi (3-chloro-4-(6-hydroxyquinolin-2-yl)benzoic acid) was assessed. Multiple doses of 3-chloro-4-(6-hydroxyquinolin-2-yl)benzoic acid were used (1, 10, 30, and 100 μM) as well as different time points (2, 4, 8, 24, and 48 h). Overall, CF HBE cells treated with a CFTR corrector (3-{6-{[1-(2,2-difluoro-1,3-benzodioxol-5-yl)cyclopropanecarbonyl]amino}-3-methylpyridin-2-yl}benzoic acid, 3 μM, 48 h) and potentiator (N-(2,4-Di-tert-butyl-5-hydroxyphenyl)-4-oxo-1,4-dihydroquinoline-3-carboxamide, 3 μM, 48 h) recorded a rapid “downward slope” post forskolin activation. When CF HBE cells were treated with 3-chloro-4-(6-hydroxyquinolin-2-yl)benzoic acid (optimal at 30 μM for 4 h) along with the CFTR corrector and CFTR potentiator, a consistent change in slope post forskolin activation was observed, suggesting increased CFTR stability in response to 3-chloro-4-(6-hydroxyquinolin-2-yl)benzoic acid treatment (FIG. 2A). The CFTR-mediated slope was calculated for all three conditions using multiple CF patient codes and Using runs (FIG. 2B). In addition, when the CFTR stimulated area under the curve (AUC) region was measured, an increase in the triple treatment (3-{6-{[1-(2,2-difluoro-1,3-benzodioxol-5-yl)cyclopropanecarbonyl]amino}-3-methylpyridin-2-yl}benzoic acid+N-(2,4-Di-tert-butyl-5-hydroxyphenyl)-4-oxo-1,4-dihydroquinoline-3-carboxamide+3-chloro-4-(6-hydroxyquinolin-2-yl)benzoic acid) AUC over DMSO negative control was observed (FIG. 2C). Also observed was an increase (40-50%) in AUC when CF HBE cells were treated with the triple combination over 3-{6-{[1-(2,2-difluoro-1,3-benzodioxol-5-yl)cyclopropanecarbonyl]amino}-3-methylpyridin-2-yl}benzoic acid+N-(2,4-Di-tert-butyl-5-hydroxyphenyl)-4-oxo-1,4-dihydroquinoline-3-carboxamide combination, suggesting added CFTR-mediated current when CF HBE cells are treated with the triple combination (3-{6-{[1-(2,2-difluoro-1,3-benzodioxol-5-yl)cyclopropanecarbonyl]amino}-3-methylpyridin-2-yl}benzoic acid/N-(2,4-Di-tert-butyl-5-hydroxyphenyl)-4-oxo-1,4-dihydroquinoline-3-carboxamide/3-chloro-4-(6-hydroxyquinolin-2-yl)benzoic acid).

Additional studies of CFTR Modulation with S-Nitrosoglutathione Reductase Inhibitor (GSNORi) 3-chloro-4-(6-hydroxyquinolin-2-yl)benzoic acid in Combination with a CFTR Corrector and with a CFTR Corrector/CFTR Potentiator

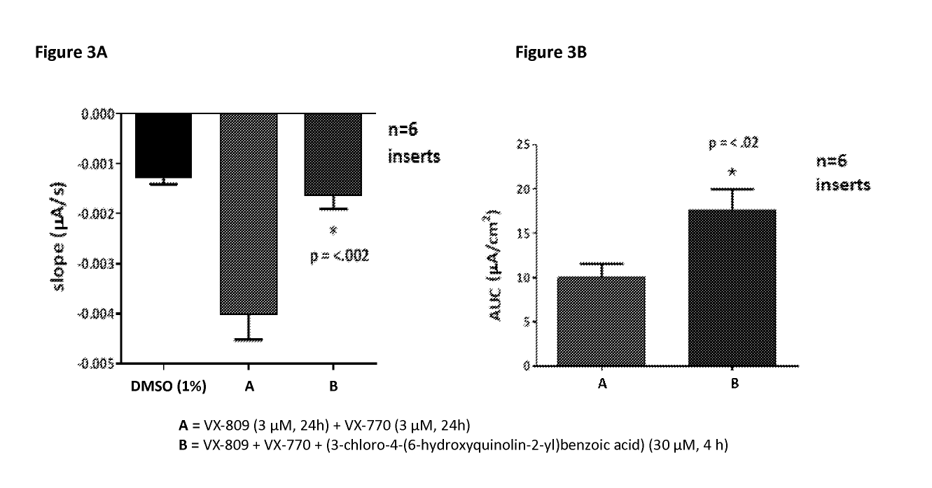

Using similar experimental approach and experimental design as described immediately above, additional data was generated with the GSNORi 3-chloro-4-(6-hydroxyquinolin-2-yl)benzoic acid in combination with a CFTR corrector VX-809 (3-{6-{[1-(2,2-difluoro-1,3-benzodioxol-5-yl)cyclopropanecarbonyl]amino}-3-methylpyridin-2-yl}benzoic acid) and in combination with VX-809 and a CFTR potentiator VX-770 (N-(2,4-Di-tert-butyl-5-hydroxyphenyl)-4-oxo-1,4-dihydroquinoline-3-carboxamide), see FIGS. 3A and B and 4A and B.

FIGS. 3A and 3B.

For data shown in FIGS. 3A and 3B, primary CF (F508del/F508del) HBE cells were grown on Snapwell™ inserts and maintained in conventional ALI media. All inserts were pretreated for 24 h with vehicle (DMSO, 0.1%), VX-809 and VX-770 (both at 3 M), or VX-809+VX-770+the GSNORi 3-chloro-4-(6-hydroxyquinolin-2-yl)benzoic acid (30 μM included for the final 4 h of the 24 h incubation). Cells were mounted in a conventional Using chamber and bathed bilaterally in Krebs Bicarbonate Ringers (KBR) solution. CF HBE cells were then sequentially exposed to the following experimental protocol: amiloride (100 μM, apical), forskolin (10 μM, bilateral), and CFTR specific inhibitor 172 (10 μM, apical).

Data shown in FIG. 3A is the slope measurements calculated from a time point immediately after the maximal forskolin Isc is achieved to a time point immediately before the addition of 172. Data shown in FIG. 3B is the area under the curve (AUC) is also quantitated as the net Cl⁻ secretion measured from a time point immediately before the addition of forskolin and a horizontal extension to a time point after the maximal 172 inhibition has been achieved. Data for FIGS. 3A and 3B represents at least 6 inserts from 2 different CF patients (CFFT010H and CFFT008G). Statistical measurements are shown between VX-809+VX-770; and VX-809+VX-770+3-chloro-4-(6-hydroxyquinolin-2-yl)benzoic acid.

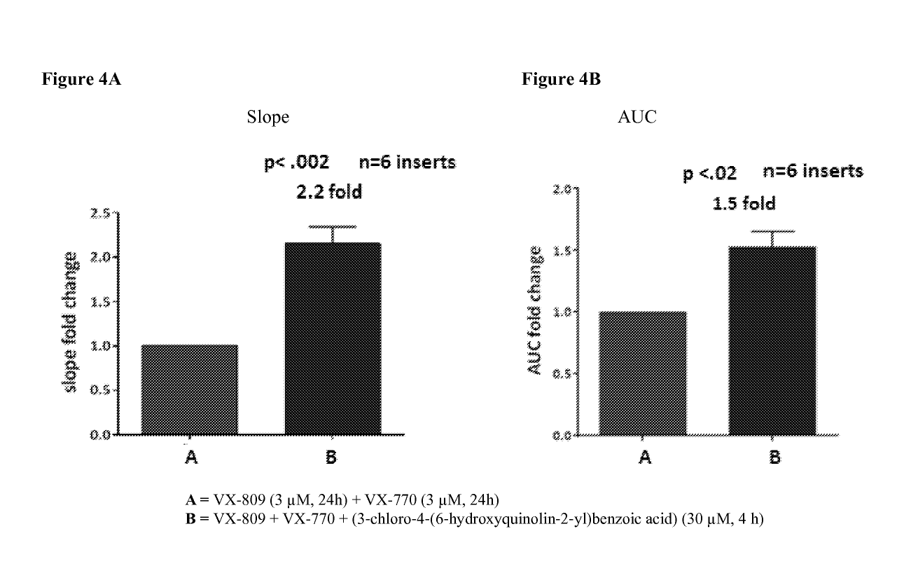

FIGS. 4A and 4B

Data from experiments of FIG. 3 is transposed into fold change by dividing the value obtained from VX-809+VX-770+3-chloro-4-(6-hydroxyquinolin-2-yl)benzoic acid treatment by the value obtained by VX-809 and VX-770 treatment alone (FIG. 4A). The slope effect is 2.2 fold greater in response with the addition of 3-chloro-4-(6-hydroxyquinolin-2-yl)benzoic acid and the AUC measurements reflect a 1.5 fold increase over VX-809/VX-770 alone (FIG. 4B).

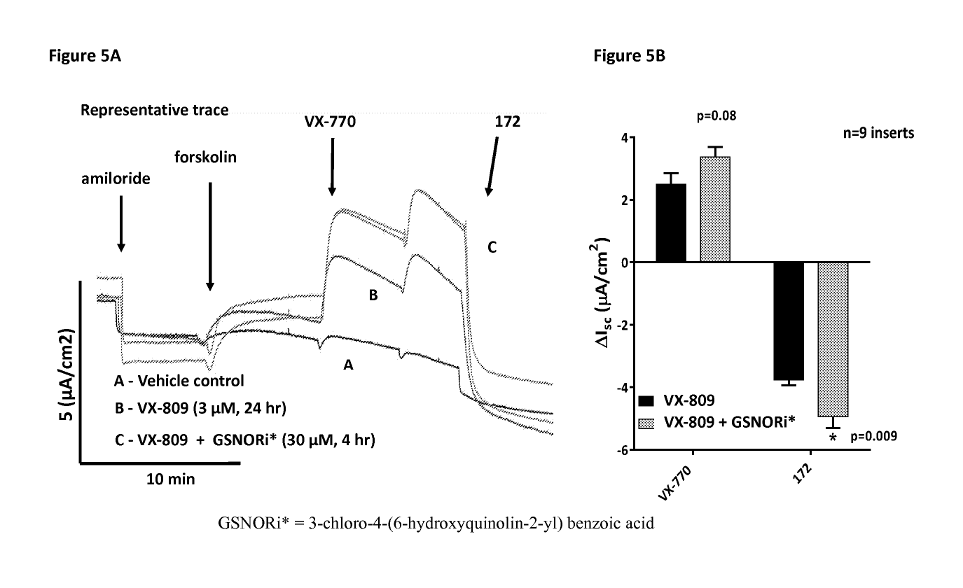

FIGS. 5A and 5B

FIG. 5A shows representative short-circuit current (Isc) tracings from CF (F508del/F508del) HBE cells grown on Snapwell™ inserts and maintained in conventional ALI media. These cells were obtained from two patient codes (CFFTKK008G and CFFTKK010H). All inserts were pretreated for 24 h with vehicle, VX-809 (3 μM), or VX-809+3-chloro-4-(6-hydroxyquinolin-2-yl)benzoic acid (30 μM for the final 4 h of the 24 h incubation; 2 examples are shown). Snapwell™ inserts are mounted in a conventional Using chamber and bathed bilaterally in Krebs Bicarbonate Ringers (KBR) solution. CF HBE cells were then sequentially exposed to the following experimental protocol: amiloride (100 μM, apical), forskolin (10 μM, bilateral), acute VX-770 (3 μM, apical), and CFTR specific inhibitor 172 (10 μM, apical).

FIG. 5A: Although there appears to be comparable stimulation of F508del CFTR in the VX-809 alone and the VX-809+3-chloro-4-(6-hydroxyquinolin-2-yl)benzoic acid combination treated inserts, it appears that more F508del CFTR is maintained and able to be potentiated by VX-770. These data suggest a nearly 30% increase in the potentiated CFTR, that approaches statistical significance (P=0.08). FIG. 5B: Furthermore the magnitude of the Isc that is inhibited by 172 is significantly greater in the CF cells exposed to VX-809+3-chloro-4-(6-hydroxyquinolin-2-yl)benzoic acid versus VX-809 alone (p<0.009). These data suggest that although the VX-809 treatment appears to be effective at promoting or correcting the F508del-CFTR and can be potentiated by VX-770, however, the addition of 3-chloro-4-(6-hydroxyquinolin-2-yl)benzoic acid results in a greater response to VX-770 and ultimately an enhanced net Cl⁻ secretion.

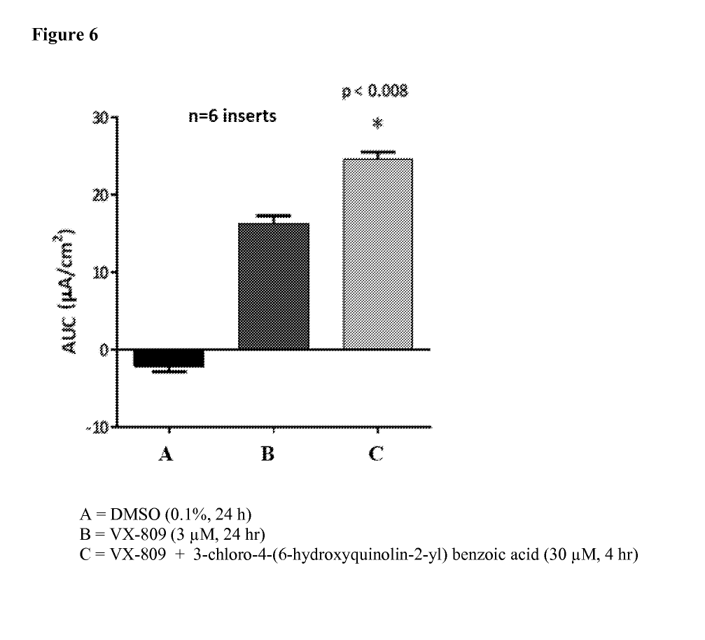

FIG. 6:

Summary data of AUC is shown in the right panel for the experiments detailed above for FIG. 5. AUC is measured as described above and a statistically significant increase (approximately a 1.5 fold increase) is observed when 3-chloro-4-(6-hydroxyquinolin-2-yl)benzoic acid is added to VX-809 compared to VX-809 alone.

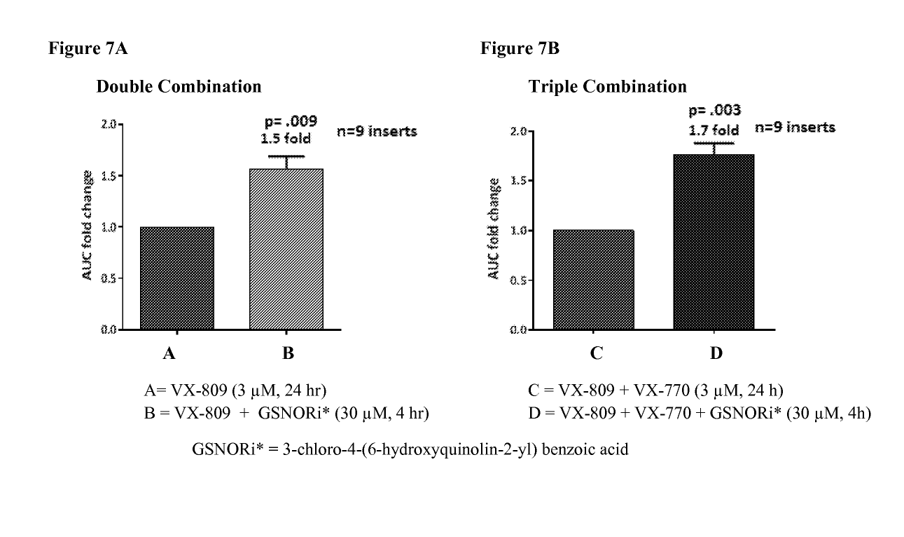

FIGS. 7A and 7B:

FIGS. 7A and 7B represent Using chamber analyses from three different patient codes (KKCFFT008G, KKCFFT010H, and KKCFFT028J) where AUC was measured from both of the conditions described above (double combination: VX-809 versus VX-809+3-chloro-4-(6-hydroxyquinolin-2-yl)benzoic acid; and triple combination: VX-809+VX-770 versus VX-809/VX-770+3-chloro-4-(6-hydroxyquinolin-2-yl)benzoic acid). At least 9 inserts were recorded and analyzed from a total of 3 codes. In all conditions, the addition of 3-chloro-4-(6-hydroxyquinolin-2-yl)benzoic acid yields a statistically significant increase in AUC that can be interpreted as a net increase in Cl⁻ secretion.

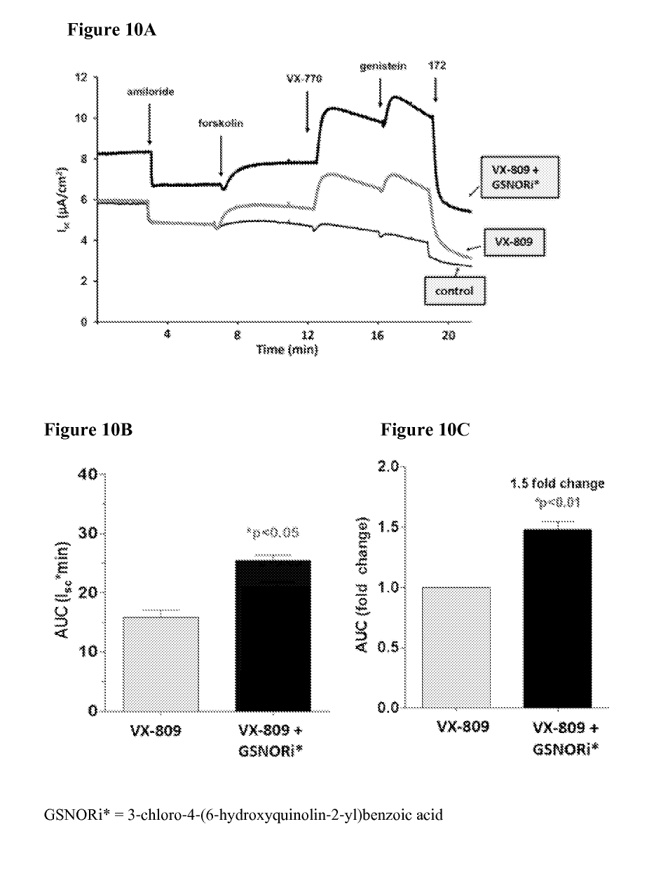

FIGS. 10A, 10B, and 10C:

Well-differentiated CF (F508del/F508del) HAE cells were mounted into Using chambers (KBR/KBR) and treated with, DMSO control, VX-809 (3 μM, 24 hr) or VX-809+GSNORi* (3-chloro-4-(6-hydroxyquinolin-2-yl) benzoic acid) (30 μM, 4 hr). FIG. 10A: A representative trace showing short circuit current (I_{sc}) is shown. FIG. 10B: The area under the curve (AUC) for total CFTR-stimulated I_{sc} was quantitated for cells treated with VX-809 alone (grey bar) or VX-809+GSNORi* (black bar). FIG. 10C: the AUC fold change was determined using different CF patient codes. (mean values are shown with SEM).

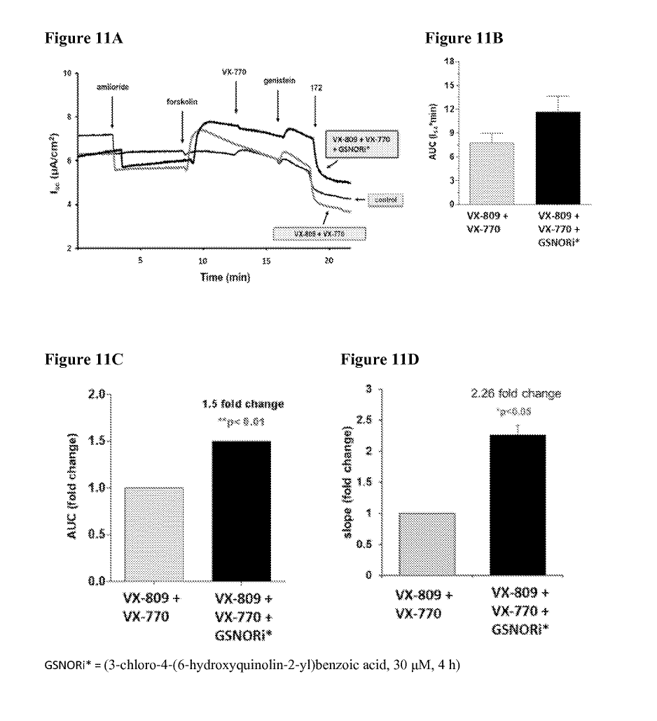

FIGS. 11A, 11B, 11C and 11D:

Well-differentiated CF (F508del/F508del) HAE cells were mounted into Using chambers (KBR/KBR) and treated with, DMSO control, VX-809+VX-770 (3 μM both) or VX-809+VX-770+GSNORi* (3-chloro-4-(6-hydroxyquinolin-2-yl) benzoic acid) (30 μM, 4 hr). FIG. 11(A): A representative trace showing short circuit current (I_{sc}) is shown. FIG. 11(B): The area under the curve (AUC) for total CFTR-stimulated I_{sc} was quantitated for cells treated with VX-809+VX-770 (grey bar) and VX-809+VX770+GSNORi* (black bar). FIG. 11(C): Fold change for AUC was quantitated such to normalize within different CF patient derived cells. FIG. 11(D): The fold change was determined for the slope of total CFTR-stimulated IL. (Fold change data obtained from different CF patients, mean values are shown with SEM).

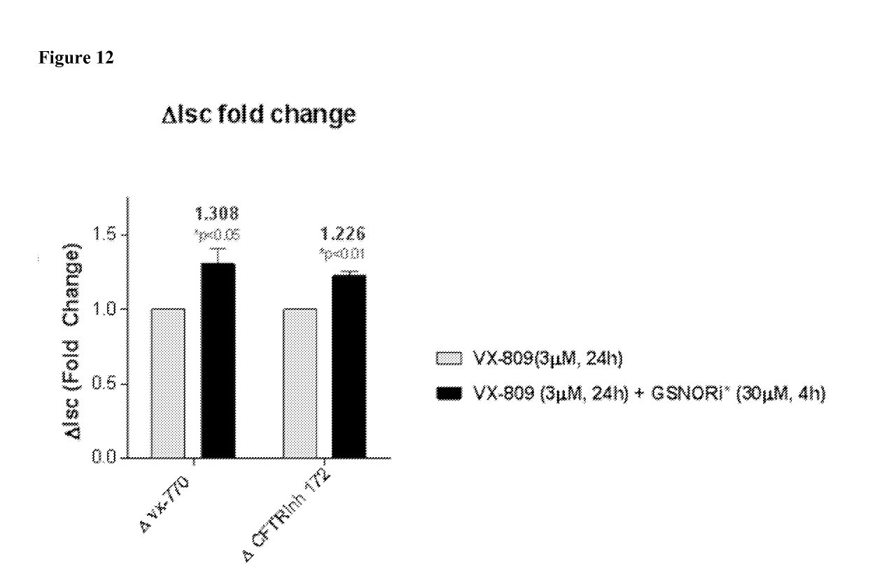

FIG. 12:

Well-differentiated CF (F508del/F508del) HAE cells were treated with either VX-809 (3 μM, 24 hr) or VX-809 (3 μM 24 hr)+GSNORi* (3-chloro-4-(6-hydroxyquinolin-2-yl) benzoic acid) (30 μM, 4 hr). I_{sc} was monitored in Using chambers at baseline and in response to addition of 100 mM amiloride (apical), 10 mM forskolin (bilateral), 3 μM vx-770 (apical), 10 mM genistein (apical), 100 mM CFTRᵢₙₕ172 (apical), and 100 mM UTP (apical). Using Chamber ΔIsc (fold change) in response to 3 μM vx-770 (apical) and 100 μM CFTRinh172 (apical) are shown in FIG. 12. Values are the mean±SEM of data from 3 experiments and 3 different patient codes.

FIGS. 10A-10C, 11A-11D and 12 provide an alternative analysis and representation of experiments used to generate FIGS. 3A-3B, 4A-4B, 5A-5B, 6, and 7A-7B.

Summary:

GSNOR inhibitor 3-chloro-4-(6-hydroxyquinolin-2-yl)benzoic acid in combination with a CFTR corrector and in combination with a CFTR corrector/potentiator resulted in increases in overall CFTR function and CFTR stability.

### Example 2: Iodide Influx Assay Measurements of CFTR Modulation with CFTR Corrector and GSNORi Combination Treatment Using FRT (F508del-CFTR_YFP) Cells

Fisher Rat Thyroid (FRT) cells were purchased from the University of San Francisco where they had transfected a construct containing F508del-CFTR and a yellow florescent protein (YFP). These cells were expanded. Semi-confluent (<95%) FRT cells were rinsed twice with a sterile phosphate buffer solution (PBS) and removed from T-75 flasks using a trypsin solution (2 mL) for 5-10 min at 37° C. in the presence of 5% CO₂. FRT media (10-12 mL, outlined below) was re-added to the flask to neutralize the trypsin. The cell suspension was collected, spun at 500 rcf for 5 min, re-suspended directly into FRT media, and counted.

FRT Media Components:
Nutrient Mixture F-123 Ham, Coon's Modification
Fetal Bovine Serum
Sodium Bicarbonate
Penicillin/Streptomycin
L-Glutamine
Zeocin
G418 (Geneticin)

FRT cells (9×10⁴, 100 μl)) were plated directly into a 96-well plate (Costar) and incubated for 2 h at 37° C. in the presence of 5% CO₂. At the end of the 2 h incubation period, cells were treated as outlined below. All compounds were prepared immediately prior to start of assay at “2×” the working concentration. Each well received the appropriate treatment solution added to the cell suspension (100 μl volume) resulting in the final appropriate concentration of compounds.

Treatment Protocol:

| Overnight (24 h) treatment | Final 1.5 h treatment |
|---|---|
| vehicle control (DMSO, 0.3%) | vehicle control (DMSO, 0.3%) |
| CFTR corrector 5-{6-[2-(2,2-difluoro-2H- | vehicle control (DMSO, 0.3%) |
| 1,3-benzodioxol-5-yl)-2- | |
| methylpropanamido]-3-methylpyridin-2- | |
| yl}thiophene-3-carboxylic acid (3 μM) | |
| CFTR corrector 5-{6-[2-(2,2-difluoro-2H- | GSNORi 3-chloro-4-(6-hydroxyquinolin-2- |
| 1,3-benzodioxol-5-yl)-2- | yl)benzoic acid (10 μM) |
| methylpropanamido]-3-methylpyridin-2- | |
| yl}thiophene-3-carboxylic acid (3 μM) | |
| CFTR corrector 5-{6-[2-(2,2-difluoro-2H- | GSNORi 3-chloro-4-(6-hydroxyquinolin-2- |
| 1,3-benzodioxol-5-yl)-2- | yl)benzoic acid (30 μM) |
| methylpropanamido]-3-methylpyridin-2- | |
| yl}thiophene-3-carboxylic acid (3 μM) | |
| CFTR corrector 5-{6-[2-(2,2-difluoro-2H- | GSNORi 3-chloro-4-(6-hydroxyquinolin-2- |
| 1,3-benzodioxol-5-yl)-2- | yl)benzoic acid (100 μM) |
| methylpropanamido]-3-methylpyridin-2- | |
| yl}thiophene-3-carboxylic acid (3 μM) | |
| CFTR corrector 5-{6-[2-(2,2-difluoro-2H- | GSNORi 3-chloro-4-(6-hydroxyquinolin-2- |
| 1,3-benzodioxol-5-yl)-2- | yl)benzoic acid (300 μM) |
| methylpropanamido]-3-methylpyridin-2- | |
| yl}thiophene-3-carboxylic acid (3 μM) | |

Following incubation (24 h), FRT cells were rinsed twice with PBS. To modulate CFTR activity, cells were stimulated with a solution (75 μl) containing forskolin (20 μM) and N-(2,4-Di-tert-butyl-5-hydroxyphenyl)-4-oxo-1,4-dihydroquinoline-3-carboxamide (3 μM). To verify that the results observed (% YFP quenching) were the result of increased CFTR function, the CFTR specific inhibitor (CFTRinh-172, 10 μM) was also added in the stimulating solution to half of the wells. All stimulated cells were then treated with iodide buffer (150 μl) and YFP quenching measurements were immediately recorded using the FlexStation III plate reader (Molecular Devices). The reader was configured with 96 well pipetting head and excitation/emission filters of 500 nm and 540 nm respectively. Fluorescent data generated for each compound was analyzed using SoftMax® Pro GXP software (Molecular Devices). F508del-CFTR activity was quantified as a percentage of the difference in normalized relative fluorescence units (RFU). The values are also defined as the percentage difference between raw RFU at initial time point (t=17s, “t₀”) and raw RFU values at final time point (“t=23, “t_{f}”). Statistical analyses were performed using Graph Pad Prism software.

Iodide Influx Assay Results:

FRT cells containing the F508del-CFTR construct showed increased YFP quenching (1.99 fold) when treated with CFTR corrector 5-{6-[2-(2,2-difluoro-2H-1,3-benzodioxol-5-yl)-2-methylpropanamido]-3-methylpyridin-2-yl}thiophene-3-carboxylic acid for 24 h over the DMSO/negative control, suggesting increased CFTR expression and/or membrane activity. The increase in % YFP quenching was markedly inhibited when FRT cells were exposed to the CFTR inhibitor, suggesting YFP quenching was indeed CFTR-mediated.

When FRT cells were exposed overnight to CFTR corrector 5-{6-[2-(2,2-difluoro-2H-1,3-benzodioxol-5-yl)-2-methylpropanamido]-3-methylpyridin-2-yl}thiophene-3-carboxylic acid followed by acute exposure to GSNORi compound 3-chloro-4-(6-hydroxyquinolin-2-yl)benzoic acid, we saw an additive dose dependent increase in % YFP quenching/CFTR activity over CFTR corrector compound 5-{6-[2-(2,2-difluoro-2H-1,3-benzodioxol-5-yl)-2-methylpropanamido]-3-methylpyridin-2-yl}thiophene-3-carboxylic acid treatment alone. There was an increase trend observed when FRT cells were treated with GSNORi 3-chloro-4-(6-hydroxyquinolin-2-yl)benzoic acid (10 and 30 μM)+CFTR corrector compound 5-{6-[2-(2,2-difluoro-2H-1,3-benzodioxol-5-yl)-2-methylpropanamido]-3-methylpyridin-2-yl}thiophene-3-carboxylic acid (2.13 and 2.15 fold increase respectively). Moreover, a further increase in % YFP quenching was observed with GSNORi 3-chloro-4-(6-hydroxyquinolin-2-yl)benzoic acid (100 and 300 μM) and CFTR corrector compound 5-{6-[2-(2,2-difluoro-2H-1,3-benzodioxol-5-yl)-2-methylpropanamido]-3-methylpyridin-2-yl}thiophene-3-carboxylic acid combination treatment over DMSO negative control treated cells (3.09 and 3.19 respectively). Overall fold increase in % YFP quenching was also observed with combination treatment (CFTR corrector compound 5-{6-[2-(2,2-difluoro-2H-1,3-benzodioxol-5-yl)-2-methylpropanamido]-3-methylpyridin-2-yl}thiophene-3-carboxylic acid+GSNORi, 3-chloro-4-(6-hydroxyquinolin-2-yl)benzoic acid, 100 and 300 μM) over corrector compound 5-{6-[2-(2,2-difluoro-2H-1,3-benzodioxol-5-yl)-2-methylpropanamido]-3-methylpyridin-2-yl}thiophene-3-carboxylic acid alone (1.55 and 1.60). The increase in % YFP quenching observed when FRT cells were treated with the combination was markedly inhibited when pre-treated with the specific CFTR inhibitor, highly suggesting modulation of CFTR activity.

Iodide Influx Assay Results:

| | | Average % | | Fold change |
|---|---|---|---|---|
| | | YFP | Fold change | vs. CFTR |
| Sample | CFTRinh172 | quenching | vs. DMSO | corrector^{†} |
| vehicle control/DMSO | no | 9.73 | 1.00 | 0.50 |
| CFTR Corrector^{†} (3 μM) | no | 19.41 | 1.99 | 1.00 |
| CFTR Corrector^{†} + | no | 20.72 | 2.13 | 1.07 |
| GSNORi* (10 μM) | | | | |
| CFTR Corrector^{†} + | no | 20.96 | 2.15 | 1.08 |
| GSNORi* (10 μM) | | | | |
| CFTR Corrector^{†} + | no | 30.10 | 3.09 | 1.55 |
| GSNORi* (30 μM) | | | | |
| CFTR Corrector^{†} + | no | 31.01 | 3.19 | 1.60 |
| GSNORi* (100 μM) | | | | |
| CFTR Corrector^{†} + | yes | 12.86 | 1.00 | 0.99 |
| GSNORi* (300 μM) | | | | |
| CFTR Corrector^{†} (3 μM) | yes | 13.04 | 1.01 | 1.00 |
| CFTR Corrector^{†} + | yes | 11.88 | 0.92 | 0.91 |
| GSNORi* (10 μM) | | | | |
| CFTR Corrector^{†} + | yes | 11.02 | 0.86 | 0.85 |
| GSNORi* (30 μM) | | | | |
| CFTR Corrector^{†} + | yes | 12.67 | 0.99 | 0.97 |
| GSNORi* (100 μM) | | | | |
| CFTR Corrector^{†} + | yes | 17.67 | 1.37 | 1.35 |
| GSNORi* (300 μM) | | | | |

CFTR Corrector^{†} = 5-{6-[2-(2,2-difluoro-2H-1,3-benzodioxol-5-yl)-2-methylpropanamido]-3-methylpyridin-2-yl}thiophene-3-carboxylic acid
GSNORi* = 3-chloro-4-(6-hydroxyquinolin-2-yl)benzoic acid

Collectively, using FRT cells transfected with the F508del-CFTR_YFP construct in the iodide influx assay, we observed overall increases in % YFP quenching/CFTR modulation when treated with the combination of CFTR corrector compound 5-{6-[2-(2,2-difluoro-2H-1,3-benzodioxol-5-yl)-2-methylpropanamido]-3-methylpyridin-2-yl}thiophene-3-carboxylic acid and GSNORi, 3-chloro-4-(6-hydroxyquinolin-2-yl)benzoic acid, over CFTR corrector alone or vehicle/DMSO negative control, highly suggesting a potential additive therapeutic benefit for combination therapy.

### Example 3: Biotinylation of Cell Surface CFTR Following Combination of CFTR Corrector and GSNORi Treatment

Fisher Rat Thyroid (FRT) cells were purchased from the University of San Francisco where they had transfected a construct containing F508del-CFTR and a yellow florescent protein (YFP). These cells were expanded for further study. Semi-confluent (<95%) FRT cells were rinsed twice with a sterile phosphate buffer solution (PBS) and removed from T-75 flasks using a trypsin solution (2 mL) for 5-10 min at 37° C. in the presence of 5% CO₂. FRT media (10-12 mL, outlined below) was re-added to the flask to neutralize the trypsin. The cell suspension was collected, spun at 500 rcf for 5 min, re-suspended directly into FRT media, and counted.

FRT Media Components:
Nutrient Mixture F-123 Ham, Coon's Modification
Fetal Bovine Serum
Sodium Bicarbonate
Penicillin/Streptomycin
L-Glutamine
Zeocin
G418 (Geneticin)

FRT cells (8×10⁵) were plated onto 6-well plates in the presence of the CFTR corrector (N1785, 3 μM). Cells were incubated for 24 h at 37° C. in the presence of 5% CO₂. The following day, FRT media was removed and cells were rinsed twice with PBS and treated as follows:
vehicle control (DMSO)
DMSO (0.3%, 24 h)+GSNORi 3-chloro-4-(6-hydroxyquinolin-2-yl)benzoic acid (100 μM, 1-2 h)
CFTR corrector 5-{6-[2-(2,2-difluoro-2H-1,3-benzodioxol-5-yl)-2-methylpropanamido]-3-methylpyridin-2-yl}thiophene-3-carboxylic acid (3 μM, 24 h)+DMSO (0.3%, 1-2 h)
CFTR corrector 5-{6-[2-(2,2-difluoro-2H-1,3-benzodioxol-5-yl)-2-methylpropanamido]-3-methylpyridin-2-yl}thiophene-3-carboxylic acid (3 μM, 24 h)+GSNORi 3-chloro-4-(6-hydroxyquinolin-2-yl)benzoic acid (100 μM, 1-2 h)

To start the biotinylation step, FRT cells were rinsed twice with PBS and treated with sodium periodate (1 mM). Plates were wrapped in foil (light sensitive) and incubated for 30 min on a plate rocker at 4° C. Solution containing sodium periodate was removed and cells were rinsed twice with PBS. Each well received a PBS solution containing EZ-link Hydrazide-LC biotin (100 μM)+analine (10 mM). Plates were covered in foil and incubated for 90 min on a rocker at 4° C. At the end of the incubation period, cells were rinsed three times in a PBS solution containing glycine (100 mM). After last wash, FRT cells were lysed with 0.1% NP-40 buffer, placed on ice for 30 min, scraped, sonicated for 5 sec, and collected in microcentrifuge tube. Protein assay was performed for all samples. Pre-washed magnetic beads (50 μl) were added to each tube and incubated overnight at 4° C. on a rotational mixer. Supernatants were removed after placing tubed on a magnetic stand. Samples were washed (5 min each wash) with TBS-T twice containing sodium chloride (150 mM, 250 mM). After the final wash, samples were combined with 2× sample buffer containing reducing agent. Samples were heated for 10 min at 60° C. and entire volume was loaded onto gel and Western blot analysis was performed as follows:

| | | Antibody | | |
|---|---|---|---|---|
| Antibody | Blocking buffer | species | antibody dilution | Incubation time |
| CFTR: | 5% dry milk/TBS-T | Mouse | 1:5,000 in 5% dry | Overnight @ 4° C. |
| UNC596 | | | milk/TBS-T | |
| Na+/K+ | 5% dry milk/TBS-T | Mouse | 1:2,000 in 5% dry | 1 h @ room temp |
| ATPase | | | milk/TBS-T | |
| NF-kB, | 5% dry milk/TBS-T | rabbit | 1:2,000 in 5% dry | 1 h @ room temp |
| p65 | | | milk/TBS-T | |
| α-mouse | 5% dry milk/TBS-T | rabbit | 1:10,000 in 5% dry | 1 h @ room temp |
| | | | milk/TBS-T | |
| α-rabbit | 5% dry milk/TBS-T | rabbit | 1:10,000 in 5% dry | 1 h @ room temp |
| | | | milk/TBS-T | |

Protein detection was performed using West Femto ECL reagent.

Results:

Within this experiment, enrichment of cell surface proteins was observed as shown by the relative signal strength of cytosolic and cell surface controls (NF-kB, p65 and Na+/K+ATPase) following pull-down. An increase in mature CFTR following overnight correction with CFTR corrector 5-{6-[2-(2,2-difluoro-2H-1,3-benzodioxol-5-yl)-2-methylpropanamido]-3-methylpyridin-2-yl}thiophene-3-carboxylic acid was seen which translate into more CFTR at the cell surface of FRT cells (F508del-CFTR) after pull-down procedure. In addition, there was a slight increase in CFTR cell surface expression observed when FRT cells were treated with CFTR corrector (5-{6-[2-(2,2-difluoro-2H-1,3-benzodioxol-5-yl)-2-methylpropanamido]-3-methylpyridin-2-yl}thiophene-3-carboxylic acid, 24 h) and GSNORi (3-chloro-4-(6-hydroxyquinolin-2-yl)benzoic acid, 1-2 h), suggesting an additive benefit for mature CFTR with a combination approach.

| | | CFTR C |
|---|---|---|
| | | Band/NaK |
| | Total CFTR | ATPase |
| Treatment (pull-dowm) | (fold change) | (fold change) |
| Vehicle control | 1.00 | 0.14 |
| CFTR Corrector 5-{6-[2-(2,2-difluoro-2H- | 8.34 | 1.00 |
| 1,3-benzodioxol-5-yl)-2- | | |
| methylpropanamido]-3-methylpyridin-2- | | |
| yl}thiophene-3-carboxylic acid (3 μM, 24 h) | | |
| GSNORi 3-chloro-4-(6-hydroxyquinolin-2- | 1.78 | 0.21 |
| yl)benzoic acid (100 μM, 1-2 h) | | |
| CFTR Corrector 5-{6-[2-(2,2-difluoro-2H- | 9.74 | 1.30 |
| 1,3-benzodioxol-5-yl)-2- | | |
| methylpropanamido]-3-methylpyridin-2- | | |
| yl}thiophene-3-carboxylic acid + GSNORi | | |
| 3-chloro-4-(6-hydroxyquinolin-2-yl)benzoic | | |
| acid | | |

### Example 4: Immunoprecipitation/Western Blot Detection of CFTR Expression Using CF Cells

### Experiment 1

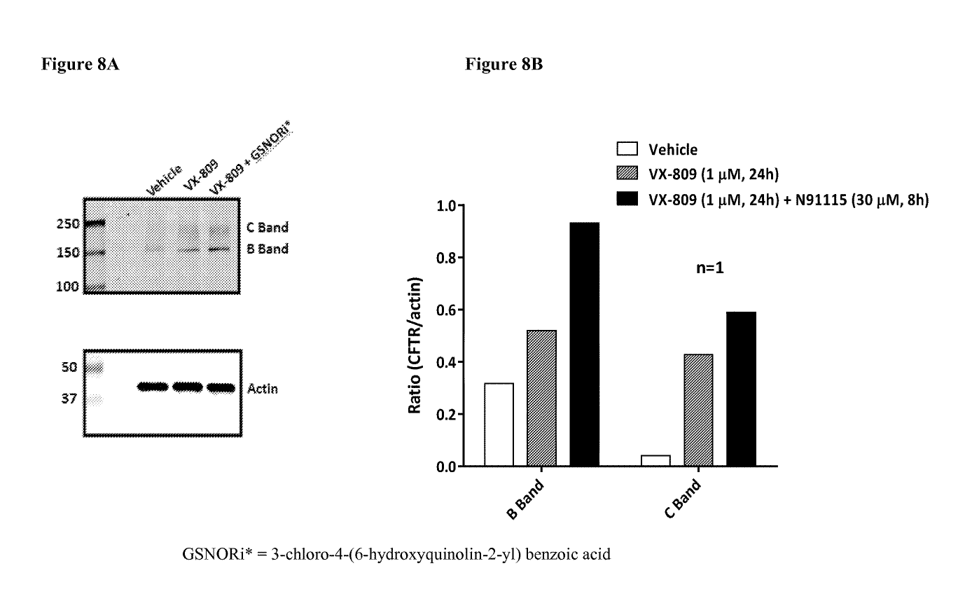

For data shown in FIGS. 8A and 8B, Fisher Rat Thyroid (FRT) cells were purchased from the University of San Francisco where they had transfected a construct containing F508del-CFTR and a yellow florescent protein (YFP). Cell media and growth protocol are outlined in Example 3.

FRT cells expressing F508del/F508del-CFTR were incubated with vehicle, VX-809 (1 μM, 24 h) or VX-809+3-chloro-4-(6-hydroxyquinolin-2-yl)benzoic acid (30 μM, last 8 h of the 24 h incubation at 37° C.). Conventional protocols (Cholon D M et al. Sci Transl Med. 2014) were used for immunoprecipitation (IP) and Western blot analysis to detect both Band B (immature) and Band C (mature) using CFTR Ab #596. As shown in FIG. 8A, as well as the quantified histobars in FIG. 8B, an increase in both CFTR band B and C are detected when cells are treated with the combination of VX-809+3-chloro-4-(6-hydroxyquinolin-2-yl)benzoic acid compared to VX-809 alone.

### Experiment 2

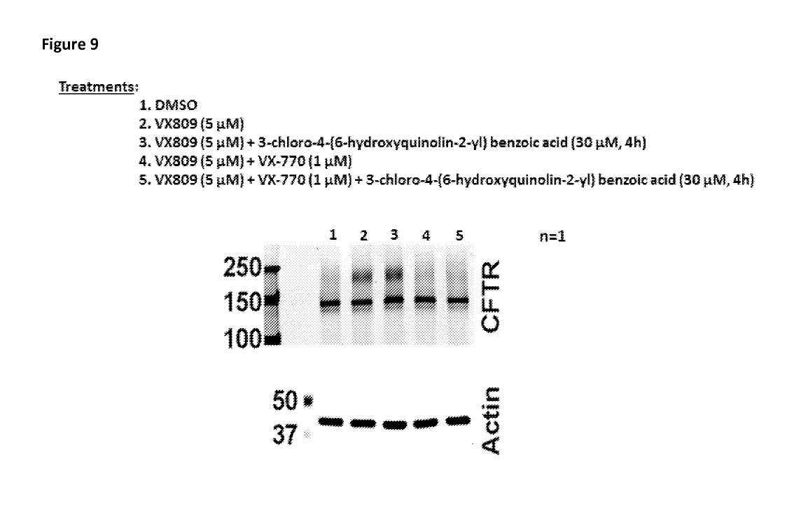

For data shown in FIG. 9, primary human CF (F508del/F508del) airway epithelial (HAE) cells were obtained, expanded, and cultured as outlined in Example 1. CF HAE cells were incubated with vehicle (DMSO), VX-809 (5 μM, 24 h), or VX-809+3-chloro-4-(6-hydroxyquinolin-2-yl)benzoic acid (30 μM, for the last 4 h of the 24 h incubation) as shown in FIG. 9. Conventional protocols were used for Western blot analysis to detect modulation of CFTR expression (Band B, immature; and Band C, mature) using the CFTR Ab #596. Preliminary data shows an increase in both band B and C were detected following the addition of VX-809+3-chloro-4-(6-hydroxyquinolin-2-yl)benzoic acid (lane 3) compared to VX-809 alone (lane 2).

### Experiment 3

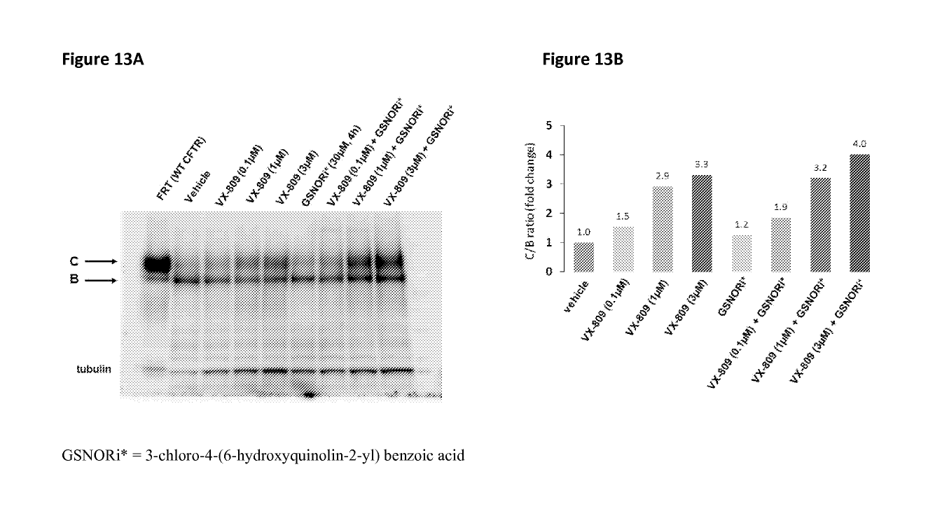

For FIG. 13, Fisher rat thyroid (FRT) cells expressing F508del/F508del-CFTR were incubated with Vehicle (DMSO), GSNORi* (3-chloro-4-(6-hydroxyquinolin-2-yl) benzoic acid, 30 μM, added at last 4 h of 24 h incubation) alone, VX-809 (0.1, 1 or 3 μM, 24 h incubation) alone, or VX-809+GSNORi* (3-chloro-4-(6-hydroxyquinolin-2-yl) benzoic acid, 30 μM, added at last 4 h of 24 h incubation). Lysates prepared in RIPA Buffer were separated on a 3-8% gradient SDS-PAGE gel (Invitrogen) and then transferred to a PVDF membrane (Invitrogen) for Western blot analysis. Blots were probed with mouse monoclonal anti-CFTR antibodes (CFTR Ab#596, UNC) followed by goat anti-mouse IgG antibodies (Santa Cruz Biotechnology). Tubulin protein was used as a loading control. Protein bands were visualized using the Biorad ChemiDoc™ system and analyzed using Image Studio software. As shown in FIG. 13A, as well as in the quantified histobars in FIG. 13B, an increase in CFTR C-band/B-band ratio (fold change) was detected in cells treated with the combination of VX-809+(3-chloro-4-(6-hydroxyquinolin-2-yl) benzoic acid) compared to VX-809 alone.

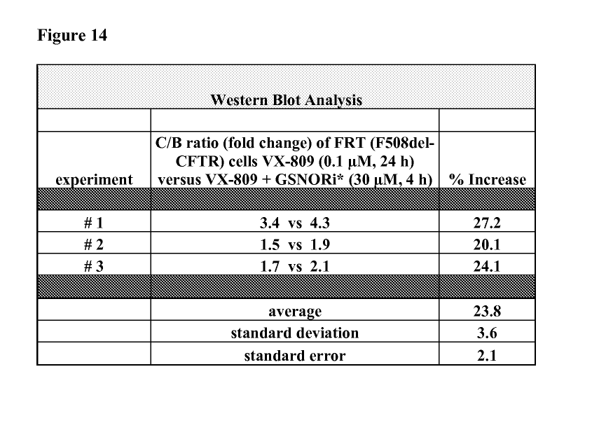

The summary table for three sets of experiments is shown in FIG. 14 representing changes in CFTR C-band/B-band ratio (fold change) when FRT (F508del-CFTR) cells were treated with VX-809 (0.1 μM, 24 h) alone compared to VX-809+(3-chloro-4-(6-hydroxyquinolin-2-yl) benzoic acid, added for the last 4 h of incubation). The table includes the data from the representative gel shown in FIG. 13. The percent increase of CFTR C-band/B-band ratios were also calculated. The average of the data shows an increase in CFTR C-band/B-band ratios with the combination of VX-809+(3-chloro-4-(6-hydroxyquinolin-2-yl) benzoic acid) compared to VX-809 alone.

### Example 5: Effects of CFTR Expression at the Plasma Membrane

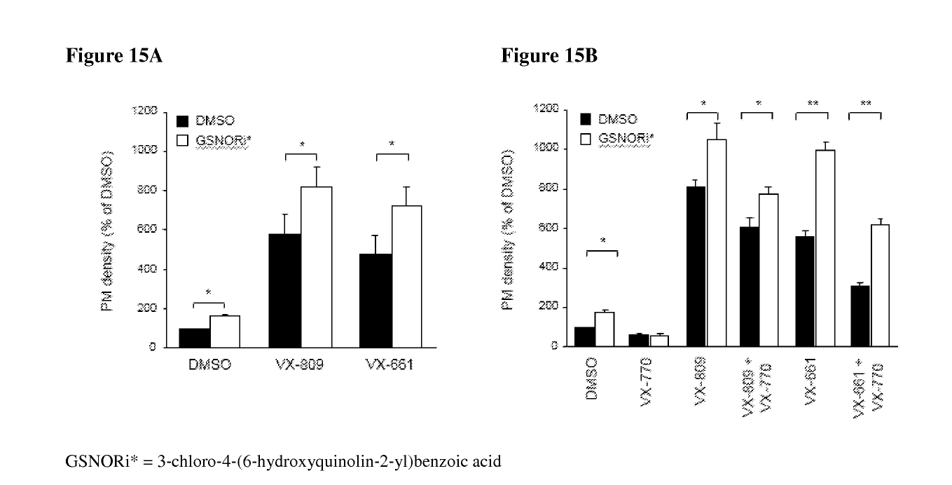

Steady state F508del-CFTR plasma membrane (PM) expression was evaluated using the CFBE41o- cell line stably expressing F508del-CFTR containing a 3HA extracellular tag. Experiments were performed by the Dr. Guido Veit and Dr. Geregely Lukacs labs, McGill University, Montreal, Quebec, Canada. Cells were grown to confluence for at least 3 days to differentiation. In FIG. 15A, steady state F508del-CFTR PM expression was measured using cells treated with VX-809 (3 μM, 24 h) or VX-661 (3 μM, 24 h) alone or in combination with 3-chloro-4-(6-hydroxyquinolin-2-yl) benzoic acid (GSNORi*) (100 μM, 24 h). The data showed a significant increase in F508del-CFTR PM expression when cells were treated with the combination of either VX-809 or VX-661 plus GSNORi*compared to the CFTR corrector alone. Moreover, FIG. 15B shows the effects of steady state PM F508del-CFTR when CFBE14o- cells were treated with a combination of VX-809, VX-661, or GSNORi* plus a potentiator (VX-770, 1 μM, 24 h). Similar to FIG. 15A, GSNORi* plus a corrector compound showed an increase in PM F508del-CFTR over either corrector compound alone. Moreover, cells treated with a corrector+potentiator showed a marked decreased in PM F508del-CFTR. This decrease was partially rescued back to corrector level alone when GSNORi* was added, suggesting potential positive GSNORi*-mediated effects on stabilizing F508del-CFTR at the PM. PM densities were determined by cell surface ELISA and are depicted as percentage of DMSO treated controls. Values show means±SEM (n=3), *−p<0.05; **−p<0.01

It will be apparent to those skilled in the art that various modifications and variations can be made in the methods and compositions of the present invention without departing from the spirit or scope of the invention.

1. A method of treating or lessening the severity of CF, comprising the step of administering to a patient in need an effective amount of
i) a GSNOR inhibitor of Formula I 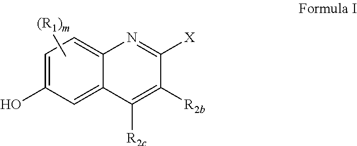
wherein
m is selected from the group consisting of 0, 1, 2, and 3;
R₁ is independently selected from the group consisting of chloro, fluoro, bromo, cyano, and methoxy;
R_{2b} and R_{2c} are independently selected from the group consisting of hydrogen, halogen, C₁-C₃ alkyl, fluorinated C₁-C₃ alkyl, cyano, C₁-C₃ alkoxy, and N(CH₃)₂;
X is selected from the group consisting of 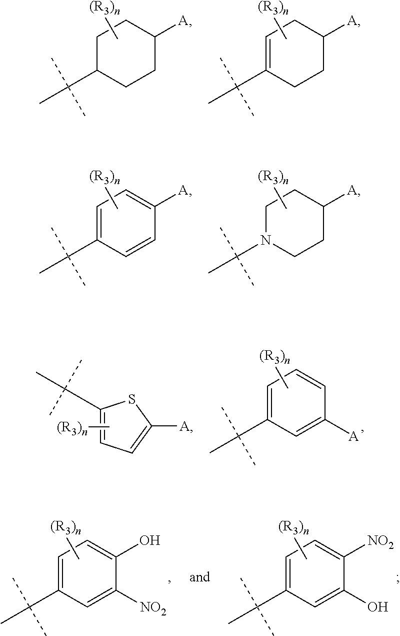
n is selected from the group consisting of 0, 1, and 2;
R₃ is independently selected from the group consisting of halogen, C₁-C₃ alkyl, fluorinated C₁-C₃ alkyl, cyano, C₁-C₃ alkoxy, and NR₄R_{4′} where R₄ and R_{4′} are independently selected from the group consisting of C₁-C₃ alkyl, or R₄ when taken together with R_{4′} form a ring with 3 to 6 members; and
A is selected from the group consisting of 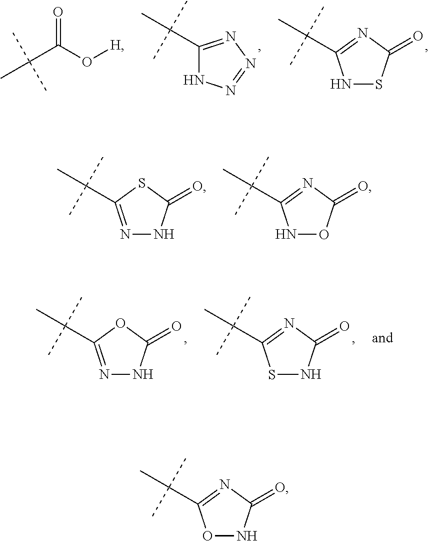
or a pharmaceutically acceptable salt thereof, and
ii) one or more secondary active agents selected from the group consisting of CFTR correctors and CFTR potentiators or pharmaceutically acceptable salt(s) thereof.

2. The method of claim 1, wherein the GSNOR inhibitor is selected from Formula I wherein R₁ is independently selected from the group consisting of chloro, fluoro, and bromo; R₃ is independently selected from the group consisting of halogen, C₁-C₃ alkyl, fluorinated C₁-C₃ alkyl, cyano, C₁-C₃ alkoxy, and NR₄R_{4′} where R₄ and R_{4′} are independently selected from the group consisting of C₁-C₃ alkyl, or R₄ when taken together with R_{4′} form a ring with 3 to 6 members; and
X is selected from the group consisting of

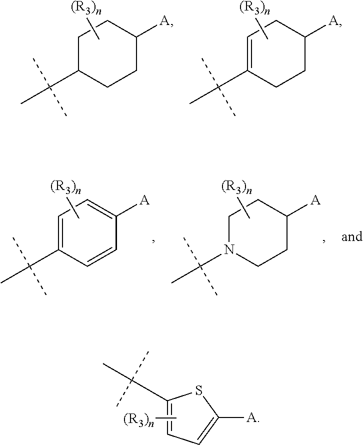

3. The method of claim 1, wherein the GSNOR inhibitor is selected from Formula I wherein R₃ is independently selected from the group consisting of halogen, C₁-C₃ alkyl, fluorinated C₁-C₃ alkyl, cyano, C₁-C₃ alkoxy, and NR₄R_{4′} where R₄ and R_{4′} are methyl, or alternatively together with the said N form the ring aziridin-1-yl or morpholino.

4. The method of claim 1, wherein the GSNOR inhibitor is selected from Formula I wherein m is selected from the group consisting of 0 and 1; R_{2b} and R_{2c} are independently selected from the group consisting of hydrogen, chloro, fluoro, methyl, trifluoromethyl, cyano, methoxy, and N(CH₃)₂; n is selected from the group consisting of 0 and 1; and R₃ is independently selected from the group consisting of fluoro, chloro, bromo, methyl, trifluoromethyl, cyano, hydroxy, methoxy, and N(CH₃)₂.

5. The method of claim 1, wherein the GSNOR inhibitor is selected from Formula I wherein X is

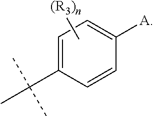

6. The method of claim 1, wherein the GSNOR inhibitor is selected from Formula I wherein A is COOH.

7. The method of claim 1, wherein the GSNOR inhibitor is a compound of Formula I wherein the compound is selected from:
4-(6-hydroxy-3-methylquinolin-2-yl)benzoic acid;
2-(4-(1H-tetrazol-5-yl)phenyl)-3-methylquinolin-6-ol;
4-(6-hydroxyquinolin-2-yl)benzoic acid;
2-(4-(1H-tetrazol-5-yl)phenyl)quinolin-6-ol;
1-(6-hydroxyquinolin-2-yl)piperidine-4-carboxylic acid;
(1r,4r)-4-(6-hydroxyquinolin-2-yl)cyclohexanecarboxylic acid;
(1s,4s)-4-(6-hydroxyquinolin-2-yl)cyclohexanecarboxylic acid;
3-chloro-4-(6-hydroxyquinolin-2-yl)benzoic acid;
2-chloro-4-(6-hydroxyquinolin-2-yl)benzoic acid;
2-fluoro-4-(6-hydroxyquinolin-2-yl)benzoic acid;
2-(4-(2H-tetrazol-5-yl)phenyl)-4-chloroquinolin-6-ol;
3-(4-(6-hydroxyquinolin-2-yl)phenyl)-1,2,4-oxadiazol-5 (2H)-one;
3-fluoro-4-(6-hydroxyquinolin-2-yl)benzoic acid;
4-(6-hydroxyquinolin-2-yl)-3-methoxybenzoic acid;
5-(6-hydroxyquinolin-2-yl)thiophene-2-carboxylic acid;
4-(6-hydroxyquinolin-2-yl)cyclohex-3-enecarboxylic acid;
4-(3-fluoro-6-hydroxyquinolin-2-yl)benzoic acid;
4-(4-chloro-3-fluoro-6-hydroxyquinolin-2-yl)benzoic acid;
4-(3-chloro-6-hydroxyquinolin-2-yl)benzoic acid;
3-(2-fluoro-4-(6-hydroxyquinolin-2-yl)phenyl)-1,2,4-oxadiazol-5(4H)-one;
3-(3-fluoro-4-(6-hydroxyquinolin-2-yl)phenyl)-1,2,4-oxadiazol-5(4H)-one;
4-(4-chloro-6-hydroxyquinolin-2-yl)benzoic acid;
2-(2-chloro-4-(2H-tetrazol-5-yl)phenyl)quinolin-6-ol;
5-(4-(6-hydroxyquinolin-2-yl)phenyl)-1,3,4-oxadiazol-2(3H)-one;
3-(dimethylamino)-4-(6-hydroxyquinolin-2-yl)benzoic acid;
4-(4-fluoro-6-hydroxyquinolin-2-yl)benzoic acid;
4-(6-hydroxyquinolin-2-yl)-3-methylbenzoic acid;
4-(3-chloro-6-hydroxyquinolin-2-yl)-3-fluorobenzoic acid;
3-(4-(6-hydroxyquinolin-2-yl)phenyl)-1,2,4-thiadiazol-5(2H)-one;
4-(6-hydroxyquinolin-2-yl)-3-(trifluoromethyl)benzoic acid;
4-(6-hydroxy-3-(trifluoromethyl)quinolin-2-yl)benzoic acid;
2-(4-carboxyphenyl)-6-hydroxyquinoline 1-oxide;
5-(4-(6-hydroxyquinolin-2-yl)phenyl)-1,3,4-thiadiazol-2(3H)-one;
5-(4-(6-hydroxyquinolin-2-yl)phenyl)-1,2,4-oxadiazol-3 (2H)-one;
(1r,4r)-4-(3-chloro-6-hydroxyquinolin-2-yl)cyclohexanecarboxylic acid;
(1s,4s)-4-(3-chloro-6-hydroxyquinolin-2-yl)cyclohexanecarboxylic acid;
3-chloro-4-(4-fluoro-6-hydroxyquinolin-2-yl)benzoic acid;
2-(5-(2H-tetrazol-5-yl)thiophen-2-yl)quinolin-6-ol;
5-(4-(6-hydroxyquinolin-2-yl)phenyl)-1,2,4-thiadiazol-3 (2H)-one;
3-fluoro-4-(4-fluoro-6-hydroxyquinolin-2-yl)benzoic acid;
1-(6-hydroxy-3-(trifluoromethyl)quinolin-2-yl)piperidine-4-carboxylic acid;
4-(5-chloro-6-hydroxyquinolin-2-yl)benzoic acid;
(1r,4r)-4-(6-hydroxy-3-(trifluoromethyl)quinolin-2-yl)cyclohexanecarboxylic acid;
(1s,4s)-4-(6-hydroxy-3-(trifluoromethyl)quinolin-2-yl)cyclohexanecarboxylic acid;
4-(5-bromo-6-hydroxyquinolin-2-yl)benzoic acid;
3-bromo-4-(6-hydroxyquinolin-2-yl)benzoic acid;
4-(4-(dimethylamino)-6-hydroxyquinolin-2-yl)benzoic acid;
4-(4-fluoro-6-hydroxyquinolin-2-yl)-3-methoxybenzoic acid;
3-cyano-4-(6-hydroxyquinolin-2-yl)benzoic acid;
2-(4-carboxy-2-chlorophenyl)-6-hydroxyquinoline 1-oxide;
4-(3-cyano-6-hydroxyquinolin-2-yl)benzoic acid;
4-(5-fluoro-6-hydroxyquinolin-2-yl)benzoic acid;
4-(8-fluoro-6-hydroxyquinolin-2-yl)benzoic acid; and
3-fluoro-4-(5-fluoro-6-hydroxyquinolin-2-yl)benzoic acid.

8. The method of claim 1, wherein the GSNOR inhibitor is selected from the group consisting of 3-chloro-4-(6-hydroxyquinolin-2-yl)benzoic acid, 3-fluoro-4-(6-hydroxyquinolin-2-yl)benzoic acid, and 4-(6-hydroxyquinolin-2-yl)-3-methylbenzoic acid.

9. The method of claim 1 wherein the secondary active agent of the pharmaceutical combination is a CFTR corrector.

10. The method of claim 1 wherein the secondary active agent is a CFTR potentiator.

11. The method of claim 1 wherein the secondary active agent is selected from the group consisting of 3-{6-{[1-(2,2-difluoro-1,3-benzodioxol-5-yl)cyclopropanecarbonyl]amino}-3-methylpyridin-2-yl}benzoic acid, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, and 5-{6-[2-(2,2-difluoro-2H-1,3-benzodioxol-5-yl)-2-methylpropanamido]-3-methylpyridin-2-yl}thiophene-3-carboxylic acid.

12. The method of claim 1 wherein the secondary active agent is N-(2,4-Di-tert-butyl-5-hydroxyphenyl)-4-oxo-1,4-dihydroquinoline-3-carboxamide.

13. The method of claim 1 wherein the secondary active agents are a CFTR corrector and a CFTR potentiator.

14. The method of claim 13 wherein consists of two secondary active agents wherein the first is the CFTR potentiator N-(2,4-Di-tert-butyl-5-hydroxyphenyl)-4-oxo-1,4-dihydroquinoline-3-carboxamide, and the second is a CFTR corrector selected from the group consisting of 3-{6-{[1-(2,2-difluoro-1,3-benzodioxol-5-yl)cyclopropanecarbonyl]amino}-3-methylpyridin-2-yl}benzoic acid, 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide, and 5-{6-[2-(2,2-difluoro-2H-1,3-benzodioxol-5-yl)-2-methylpropanamido]-3-methylpyridin-2-yl}thiophene-3-carboxylic acid.

15. The method of claim 8 wherein the secondary active agents are N-(2,4-Di-tert-butyl-5-hydroxyphenyl)-4-oxo-1,4-dihydroquinoline-3-carboxamide and 3-{6-{[1-(2,2-difluoro-1,3-benzodioxol-5-yl)cyclopropanecarbonyl]amino}-3-methylpyridin-2-yl}benzoic acid.

16. The method of claim 8 wherein the secondary active agents are N-(2,4-Di-tert-butyl-5-hydroxyphenyl)-4-oxo-1,4-dihydroquinoline-3-carboxamide and 5-{6-[2-(2,2-difluoro-2H-1,3-benzodioxol-5-yl)-2-methylpropanamido]-3-methylpyridin-2-yl}thiophene-3-carboxylic acid.

17. The method of claim 8 wherein the secondary active agents are N-(2,4-Di-tert-butyl-5-hydroxyphenyl)-4-oxo-1,4-dihydroquinoline-3-carboxamide and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide.

18. The method of claim 15 wherein the GSNOR inhibitor is 3-chloro-4-(6-hydroxyquinolin-2-yl)benzoic acid and the secondary active agents are N-(2,4-Di-tert-butyl-5-hydroxyphenyl)-4-oxo-1,4-dihydroquinoline-3-carboxamide and 3-{6-{[1-(2,2-difluoro-1,3-benzodioxol-5-yl)cyclopropanecarbonyl]amino}-3-methylpyridin-2-yl}benzoic acid.

19. The method of claim 17 wherein the GSNOR inhibitor is 3-chloro-4-(6-hydroxyquinolin-2-yl)benzoic acid and the secondary active agents are N-(2,4-Di-tert-butyl-5-hydroxyphenyl)-4-oxo-1,4-dihydroquinoline-3-carboxamide and 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide.

20. The method of claim 11 wherein the GSNOR inhibitor is 3-chloro-4-(6-hydroxyquinolin-2-yl)benzoic acid and the secondary active agent is 3-{6-{[1-(2,2-difluoro-1,3-benzodioxol-5-yl)cyclopropanecarbonyl]amino}-3-methylpyridin-2-yl}benzoic acid.

21. The method of claim 12 wherein the GSNOR inhibitor is 3-chloro-4-(6-hydroxyquinolin-2-yl)benzoic acid and the secondary active agent is N-(2,4-Di-tert-butyl-5-hydroxyphenyl)-4-oxo-1,4-dihydroquinoline-3-carboxamide.

22. The method of claim 11 wherein the wherein the GSNOR inhibitor is 3-chloro-4-(6-hydroxyquinolin-2-yl)benzoic acid and the secondary active agent is 1-(2,2-difluoro-1,3-benzodioxol-5-yl)-N-[1-[(2R)-2,3-dihydroxypropyl]-6-fluoro-2-(2-hydroxy-1,1-dimethylethyl)-1H-indol-5-yl]-cyclopropanecarboxamide.
